(19) United States
(12) Patent Application Publication
(10) Pub. No.: US 2026/0208190 A1
(43) Pub. Date: Jul. 23, 2026

(54) DEVICES, SYSTEMS, AND METHODS FOR SEPARATION AND/OR ENRICHMENT OF PARTICLES IN SOLUTION

(71) Applicant: Duke University, Durham, NC (US)

(72) Inventors: Ty Naquin, Durham, NC (US); Tony Jun Huang, Durham, NC (US)

(73) Assignee: Duke University, Durham, NC (US)

(21) Appl. No.: 19/454,984

(22) Filed: Jan. 21, 2026

Related U.S. Application Data
(60) Provisional application No. 63/747,606, filed on Jan. 21, 2025.

(51) Int. Cl. B01L 3/00; C12Q 1/6886; G01N 21/65

(52) U.S. Cl. CPC B01L 3/502753; C12Q 1/6886; G01N 21/658; B01L 2200/0647; B01L 2200/141; B01L 2200/142; B01L 2300/0803; B01L 2300/1894; B01L 2400/0409; B01L 2400/0436; C12Q 2600/178

(57) ABSTRACT

Provided herein is a system for separation of particles in a liquid sample. The system can include a substrate, a containment structure, one or more acoustic transducers, and a disc. The containment structure can be disposed on the substrate and define a fluid-retaining region configured to contain a fluid. The one or more acoustic transducers can be coupled to substrate and configured to provide acoustic waves to the fluid such that the fluid is rotated by the acoustic waves. The disc can include a plurality of microfluidic channels. The disc can be configured to be disposed on top of the fluid and rotate with the fluid. The plurality of microfluidic channels can be configured to receive the liquid sample.

### CROSS-REFERENCE TO RELATED APPLICATIONS

This application claims priority to U.S. Provisional Application No. 63/747,606 filed Jan. 21, 2025, the contents of which are entirely incorporated by reference herein.

### STATEMENT REGARDING FEDERALLY SPONSORED RESEARCH OR DEVELOPMENT

This invention was made with government support under Federal Grant No. R01GM132603 awarded by the National Institutes of Health and Federal Grant No. DGE-2139754 awarded by the National Science Foundation. The federal government has certain rights to this invention.

### FIELD OF DISCLOSURE

The present disclosure relates to devices, systems, and methods for separation of particles in solution.

### BACKGROUND

Circulating exosomes are considered promising non-invasive biomarkers for investigating disease development, progression, and early disease detection. Exosome isolation, however, is challenging due to their small size and cohabitation with numerous other bioparticles in bodily fluids. Although several methods of separating exosomes have been developed, current techniques fail to meet research and clinical needs due to lengthy processing times, large sample consumption, and limited purity and yield.

Therefore, there is a need for devices, systems, and methods for separation and/or enrichment of particles in solution.

### SUMMARY

In some aspects, the techniques described herein relate to a system for separation of particles in a liquid sample, the system including: a substrate; a containment structure disposed on the substrate and defining a fluid-retaining region configured to contain a fluid; one or more acoustic transducers coupled to the substrate and configured to provide acoustic waves to the fluid such that the fluid is rotated by the acoustic waves; and a disc including a plurality of microfluidic channels, the disc configured to be disposed on top of the fluid and rotate with the fluid, the plurality of microfluidic channels configured to receive the liquid sample, wherein rotation of the disc establishes a centrifugal field and an induced fluid flow within the plurality of microfluidic channels to transport particles within the liquid sample along the plurality of microfluidic channels at velocities dependent on one or more of particle size and particle density, thereby effecting separation and/or enrichment of a selected particle fraction of the liquid sample.

In some aspects, the techniques described herein relate to a system, wherein the plurality of microfluidic channels are slanted channels having a first portion extending to a second portion, wherein the first portion is connected to the second portion at an angle, wherein the angle is about 15 degrees to about 90 degrees, and wherein the angle shortens a particle transport path and increases a separation rate of the particles in the liquid sample.

In some aspects, the techniques described herein relate to a system, further including at least one biosensing unit located along at least one of the plurality of microfluidic channels, wherein the biosensing unit is configured to determine a composition of the liquid sample at a location along at least one of the plurality of microfluidic channels.

In some aspects, the techniques described herein relate to a system, wherein the at least one biosensing unit includes: a surface-enhanced Raman spectroscopy region including a plurality of plasmonic nanostructures immobilized at the location along at least one of the plurality of microfluidic channels, the plurality of plasmonic nanostructures configured to provide electromagnetic field enhancement sufficient to detect target-induced spectral signatures; and an excitation light source configured to illuminate the surface-enhanced Raman spectroscopy region at an excitation wavelength sufficient to induce a reporter Raman signal, wherein the reporter Raman signal is indicative of the composition of the liquid sample in the surface-enhanced Raman spectroscopy region.

In some aspects, the techniques described herein relate to a system, wherein the at least one biosensing unit further includes a plurality of nucleic acid or nucleic acid analog probes covalently attached to the plurality of plasmonic nanostructures and configured as inverse molecular sentinel probes, that, upon hybridization with one or more biomarkers in the liquid sample, undergo a conformational change that increases the reporter Raman signal.

In some aspects, the techniques described herein relate to a system, wherein the plurality of nucleic acid or nucleic acid analog probes include multiplexed inverse molecular sentinel probes specific for at least one microRNA target.

In some aspects, the techniques described herein relate to a system, wherein the plurality of plasmonic nanostructures include bimetallic nanostars each having a gold core and a silver shell with a shell thickness configured to maximize the electromagnetic field enhancement.

In some aspects, the techniques described herein relate to a system, wherein the one or more acoustic transducers include two opposed pairs of acoustic transducers, each pair positioned on opposing sides of the fluid-retaining region.

In some aspects, the techniques described herein relate to a system, wherein the selected particle fraction includes exosomes.

In some aspects, the techniques described herein relate to a system, wherein the containment structure includes a hydrophobic ring bonded to the substrate.

In some aspects, the techniques described herein relate to a system, wherein the disc further includes a membrane configured to enclose the liquid sample within the plurality of microfluidic channels, wherein the membrane is configured to reduce evaporation and ambient contamination.

In some aspects, the techniques described herein relate to a system, further including a temperature control element thermally coupled to the substrate and configured to maintain a sample temperature within a predetermined range.

In some aspects, the techniques described herein relate to a method for separation of particles in a liquid sample, the method including: providing the liquid sample to a plurality of microfluidic channels of a disc; setting the disc on top of a fluid contained within a fluid-retaining region of a containment structure disposed on a substrate; and providing, via one or more acoustic transducers coupled to the substrate, acoustic waves to the fluid such that the fluid is rotated by the acoustic waves, thereby rotating the disc, wherein rotation of the disc establishes a centrifugal field and an induced fluid flow within the plurality of microfluidic channels to transport particles within the liquid sample along the plurality of microfluidic channels at velocities dependent on one or more of particle size and particle density, thereby effecting separation or enrichment of a selected particle fraction of the liquid sample.

In some aspects, the techniques described herein relate to a method, further including providing, via an excitation light source, an excitation light to a surface-enhanced Raman spectroscopy region along at least one of the plurality of microfluidic channels to induce a reporter Raman signal, wherein the reporter Raman signal is indicative of a composition of the liquid sample in the surface-enhanced Raman spectroscopy region.

In some aspects, the techniques described herein relate to a method, wherein the surface-enhanced Raman spectroscopy region includes a plurality of plasmonic nanostructures configured to provide electromagnetic field enhancement sufficient to detect target-induced spectral signatures.

In some aspects, the techniques described herein relate to a method, further including detecting an increase to the reporter Raman signal when a plurality of nucleic acid or nucleic acid analog probes covalently attached to the plurality of plasmonic nanostructures hybridize with one or more biomarkers in the liquid sample and undergo a conformational change.

In some aspects, the techniques described herein relate to a method, wherein the plurality of nucleic acid or nucleic acid analog probes include multiplexed inverse molecular sentinel probes specific for at least one microRNA target.

In some aspects, the techniques described herein relate to a method, wherein the plurality of nucleic acid or nucleic acid analog probes include oligonucleotide probes and the at least one microRNA target includes miR-21 and miR-221.

In some aspects, the techniques described herein relate to a method, wherein the at least one microRNA target is indicative of circulating colorectal cancer.

In some aspects, the techniques described herein relate to a method, further including providing, via a temperature control element thermally coupled to the substrate, temperature regulation to maintain a sample temperature within a predetermined range.

Other aspects and iterations of the invention are described more thoroughly below.

### BRIEF DESCRIPTION OF FIGURES

The patent or application file contains at least one drawing executed in color. Copies of this patent or patent application publication with color drawing(s) will be provided by the Office upon request and payment of the necessary fee.

The description will be more fully understood with reference to the following figures and graphs, which are presented as various embodiments of the disclosure and should not be construed as a complete recitation of the scope of the disclosure. It is noted that, for purposes of illustrative clarity, certain elements in various drawings may not be drawn to scale. Understanding that these drawings depict only exemplary embodiments of the disclosure and are not therefore to be considered limiting of its scope, the principles herein are described and explained with additional specificity and detail through the use of the accompanying drawings in which:

FIG. 1 illustrates a system for separation and/or enrichment of particles in solution in one example.

FIG. 2 illustrates a system for separation and/or enrichment of particles in solution in one example.

FIG. 3 illustrates a system for separation and/or enrichment of particles in solution in one example.

FIG. 4 illustrates a system for separation and/or enrichment of particles in solution in one example.

FIG. 5 illustrates a system for separation and/or enrichment of particles in solution in one example.

FIG. 6 is a graph of disc rotation speed in relation to acoustic transducer offset distance.

FIG. 7 is a graph of disc rotation speed in relation to a droplet volume.

FIG. 8 is a graph of disc rotation speed in relation to input voltage.

FIG. 9 illustrates particle concentration before and after acoustically driven disc spinning.

FIG. 10 illustrates nanoparticle tracking analysis (NTA) results before and

after acoustically driven disc spinning.

FIG. 11 illustrates blood plasma separation in a straight channel and a tilted channel.

FIG. 12 is a graph of the height of separation plasma in relation to time for separation based for multiple tilt angles.

FIG. 13 illustrates an overlay of microscopic images showing the experimental results of particle separation with 150 nm (blue) and 28 nm (green) particles. Since larger particles concentrate first, by controlling the voltage and time applied, the 150 nm particles can be gathered to the channel edge while the 28 nm particles remain dispersed throughout the channel.

FIG. 14 is a western blot analysis of the exosome biomarker CD63 and the protein biomarker Apolipoprotein A (apoA). Results demonstrate that exosomes remain present before and after acoustic processing, and that apoA is present in a significantly lower degree after processing. This result illustrates the separation of exosomes from proteins, resulting in a purer exosome sample.

FIG. 15 illustrates a range of simulated plasmonic nanostar models.

FIG. 16 illustrates maximum electric field enhancement and heat loss of all simulated models.

FIG. 17 illustrates nanostar morphology with the greatest SERS enhancement factor.

FIG. 18 illustrates absorbance spectra of all nanoparticle morphologies synthesized.

FIG. 19 illustrates HAADF-STEM images of nanaostars −1.5, −5, −10.

FIG. 20 illustrates SERS intensity at 1583 cm⁻¹ of each different nanoparticle morphology.

FIG. 21 illustrates SERS of a plasmonic nanostar region on a disc surface before and after spinning.

FIG. 22 illustrates a schematic diagram of a sample-to-answer workflow for PCR analysis.

FIG. 23 illustrates NTA results for patient samples after disc spinning.

FIG. 24 illustrates PCR results showing the relative expression level of miR21 in whole plasma (solid bars) and plasma processed through the system (hollow bars), respectively.

FIG. 25 illustrates PCR results showing the relative expression level of miR221 in whole plasma (solid bars) and plasma processed through the system (hollow bars), respectively.

FIG. 26A illustrates a scheme depicting patient sample workflow for biomarker detection using the system.

FIG. 26B illustrates representative multiplexed SERS spectra including individual iMS probe signals.

FIG. 27 illustrates representative spectra depicting miR-21 iMS probe and miR-221 iMS probe signals in response to the addition of a synthetic target.

FIG. 28 illustrates relative SERS signal increases for each iMS probe for each patient and sample analyzed, N=6 per patient.

FIG. 29 illustrates PCA analysis results of all samples analyzed, N=24 for the cancer group and N=12 for the healthy group.

FIG. 30 illustrates an ROC curve generated using the relative SERS signal increase for each iMS probe after spectral unmixing for each recorded spectra, N=24 for the cancer group, and N=12 for the healthy group.

FIG. 31 illustrates numerical simulation results investigating fluid streaming velocity within a water droplet.

FIG. 32 illustrates an experimental acoustic streaming pattern within a droplet.

FIGS. 33A-33G illustrate a series of images showcasing the streamlining pattern at different layers of the droplet. As the particles spiral down to the bottom of the droplet, the streaming pattern acts as a whirlpool that drives particles to aggregate at the center.

FIG. 34A illustrates a straight microfluidic channel and the distance traveled by particles.

FIG. 34B illustrates a tilted microfluidic channel which causes the particles to experience a shortened path length.

FIG. 35 illustrates disc rotation speed for one pair of transducers and two pairs of transducers.

FIG. 36 illustrates a droplet volume before and after 35 minutes of applied acoustics.

FIG. 37 illustrates droplet temperature before and during acoustic actuation.

FIG. 38 illustrates NTA concentration results before and after enrichment for 200 nm particles.

FIG. 39 illustrates NTA concentration results before and after enrichment for 100 nm particles.

FIG. 40 illustrates NTA concentration results before and after enrichment for 50 nm particles.

FIG. 41 illustrates separation of plasma from blood in straight and bent channels of the system.

FIG. 42 illustrates particle concentration and distribution before (blue) and after (red) processing by the system.

FIG. 43 illustrates protein concentration in plasma before and after enrichment with by the system.

FIG. 44 illustrates purity from protein contaminants of the system enriched exosome samples.

FIG. 45 illustrates a SEM image of nanostars functionalized onto the ends of a disc.

FIG. 46 illustrates a representative spectra of SH-C6-AAAAA-CY5 recorded from different sensing regions, N=8.

FIG. 47 illustrates the normalized peak height at 557 cm⁻¹ of SH-C6-AAAA-CY5 SERS spectra recorded from different sensing regions, N=20.

FIG. 48 illustrates normalized SERS intensities of each iMS probe in the “ON” and “OFF” configurations, N=10.

FIG. 49 illustrates microRNA (miRNA) and its associated functions.

FIG. 50 illustrates miRNA as a disease biomarker.

FIG. 51 illustrates normal Raman scattering.

FIG. 52 illustrates local surface plasmon resonance.

FIG. 53 illustrates surface-enhanced Raman scattering (SERS).

FIG. 54 illustrates surfactant-free gold nanostars used to leverage high aspect ratio branches to generate an intense local electric field enhancement.

FIG. 55 illustrates theoretical enhancement of bimetallic nanostars.

FIG. 56 illustrates enhancement of bimetallic nanostars.

FIGS. 57A-57C illustrate experimental validation of theoretical enhancement of bimetallic nanostars.

FIG. 58 illustrates a label-free exosome enrichment verification.

FIG. 59 illustrates a SERS-based liquid biopsy of enrichment exosomes.

FIG. 60 illustrates inverse molecular sentinel miRNA probes.

FIG. 61A is a graph of increased SERS intensity in relation to target DNA.

FIG. 61B is a graph of increased SERS intensity in relation to Raman shift.

FIG. 61C is a graph of SERS intensity in relation to time.

FIG. 62 illustrates a flowchart of a method for separation and enrichment of particles in a solution.

Reference characters indicate corresponding elements among the views of the drawings. The headings used in the figures do not limit the scope of the claims.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without parting from the spirit and scope of the disclosure. Thus, the following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be references to the same embodiment or any embodiment; and such references mean at least one of the embodiments.

Reference to “one embodiment”, “an embodiment”, or “an aspect” means that a particular feature, structure, or characteristic described in connection with the embodiment is included in at least one embodiment of the disclosure. The appearances of the phrase “in one embodiment” or “in one aspect” in various places in the specification are not necessarily all referring to the same embodiment, nor are separate or alternative embodiments mutually exclusive of other embodiments. Moreover, various features are described which may be exhibited by some embodiments and not by others.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any example term. Likewise, the disclosure is not limited to various embodiments given in this specification.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The term “substantially” is defined to be essentially conforming to the particular dimension, shape or other word that substantially modifies, such that the component need not be exact.

The terms “comprising,” “including” and “having” are used interchangeably in this disclosure. The terms “comprising,” “including” and “having” mean to include, but not necessarily be limited to the things so described.

The term “coupled” as used herein is defined as connected, whether directly or indirectly through intervening components, and is not necessarily limited to physical connections. The connection can be such that the objects are permanently connected or releasably connected, either physically or functionally.

Exosomes are cell-derived nanovesicles that have recently gained popularity as potential biomarkers for liquid biopsies due to their plethora of molecular cargo, such as nucleic acids and proteins. As a result, circulating exosomes are considered promising non-invasive biomarkers for investigating disease development, progression, and early disease detection. Exosome isolation, however, is challenging due to their small size and cohabitation with numerous other bioparticles in bodily fluids. Although several methods of separating exosomes have been developed, such as ultracentrifugation, polymer-assisted precipitation, and ultrafiltration, current techniques fail to meet research and clinical needs due to a lengthy processing time, large sample consumption, and limited purity and yield.

Clinical applications of exosomes are further hindered by limitations in biomarker analysis. Their lengthy procedures and cost-prohibitive reagent and instrumentation requirements limit conventional detection methods such as digital droplet polymerase chain reaction (PCR), benchtop RT-qPCR, sequencing, and microarray analysis. To circumvent these drawbacks, other sensing strategies have been implemented, including isothermal amplification, lateral flow tests, and electrochemical sensing assays, but these techniques lack the specificity and sensitivity needed for widespread adoption in clinical settings due to the low concentrations of biomarkers in bodily fluids. Therefore, biomarker detection remains challenging without a unified exosome enrichment and in-situ analysis platform to purify and concentrate biomarkers before detection.

Recently, microfluidics has emerged as a promising candidate for exosome separation and analysis. Current microfluidic technologies employ a variety of mechanisms such as electric fields, magnetics, and acoustics to control bioparticles and actuate fluids. However, Existing microfluidic separation technologies have difficulties manipulating particles below 50 nm in size and removing the small, non-exosomal contaminants such as lipoproteins, leading to less reliable and consistent biomarker diagnosis. Additionally, current technologies often produce relatively dilute samples and need help concentrating the exosomes, limiting the molecular information available for analysis. Furthermore, many existing microfluidic systems must be coupled with bulky analytical systems, making them unsuitable for point-of-care testing. For example, microfluidic separation often uses counting approaches such as nanoparticle tracking analysis (NTA) and dynamic light scattering. Still, these technologies have bulky fingerprints and provide little diagnostic information. Conventional molecular assays such as western blots and ELISA require large sample volumes, hampering their application in settings with large patient cohorts or limited specimen availability. Hence, there exists a need for rapid enrichment technology with integrated multiplexed biomarker sensing to bring the next-generation liquid biopsy analysis into fruition.

The system provided herein overcomes the challenges of prior exosome separation technologies. The system provided herein utilizes acoustic separation and concentration of exosomes for nucleotide detection, which allows the enrichment and detection of exosomes using a rotating microfluidic disc. In some examples, the system can further include an integrated multiplexed, plasmonic nanostar-based miRNA assay. Acoustofluidic disc rotation can be generated by coupling surface acoustic waves and the fluid layer on which the disc floats to enable centrifugation and fluid actuation within the microfluidic channels on the disc surface. The system can be used for separation and concentration of both micro and nanoscale objects. The interplay between the centrifugal and drag forces can lead to size and density-dependent transport velocity, which are utilized to separate the relatively large exosomes from protein contaminants in plasma. Additionally, a bimetallic nanostar substrate can be functionalized on the disc surface to verify the enrichment mechanism through label-free Surface-enhanced Raman scattering (SERS) sensing of the concentrated exosome sample.

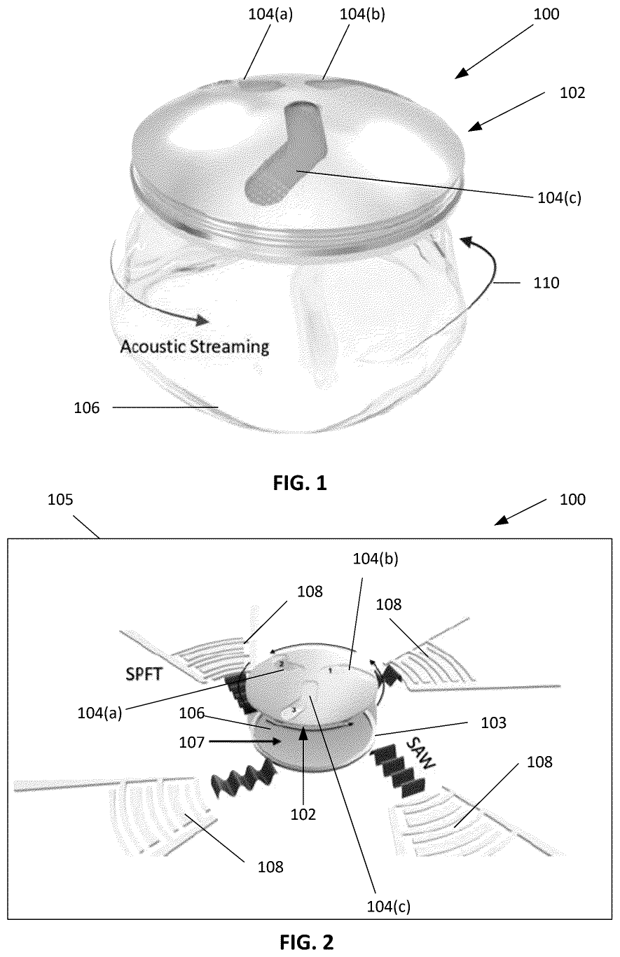

FIGS. 1-3 illustrate a system 100 for separation and/or enrichment of particles in a solution (e.g., liquid solution). In some examples, the liquid solution can include a sample from a patient (e.g., blood, saliva, or other bodily fluids). The system 100 can include a substrate 105. The substrate 105 can be operable to have components of the system 100 attached thereto. In some examples, the substrate 105 can include a piezoelectric material. In some examples, the substrate 105 can include a lithium niobate substrate. In some examples, the substrate 105 can include a 128° Y-cut LiNbO₃ wafer.

The system 100 can include a containment structure 103. The containment structure 103 can be disposed on the substrate 105. The containment structure 103 can be configured to contain a fluid 106. In some examples, the containment structure 103 can be coupled to the substrate 105. For example, the containment structure 103 can be coupled to the substrate 105 via an oxygen plasma treatment followed by heating. Other techniques for coupling the containment structure 103 to the substrate 105 can be used.

The containment structure 103 can define a fluid-retaining region 107 configured to retain a fluid 106. In some examples, the containment structure 103 can be a ring having side walls defining a fluid-retaining region 107. In some examples, containment structure 103 can include a hydrophobic ring. For example, a hydrophobic ring can repel the fluid 106 such that the fluid 106 can move freely within the fluid-retaining region 107. In some examples, the hydrophobic ring can include a polymeric ring. In some examples, the containment structure 103 can include a polydimethylsiloxane (PDMS) ring. In some examples, the containment structure 103 can have an open top end. In some examples, the fluid 106 can include water (e.g., water droplet). In other examples, other fluids can be used. In some examples, the fluid 106 is a low viscosity fluid, such that the fluid 106 can be easily rotated by acoustic waves.

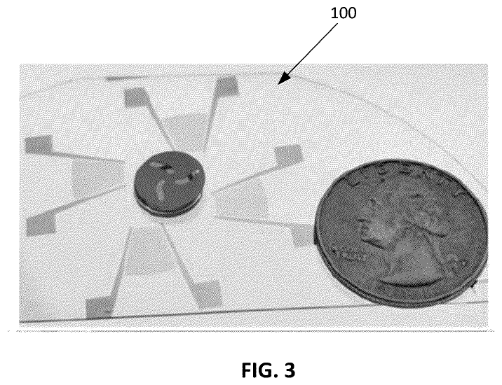

The system 100 can include one or more acoustic transducers 108. In some examples, the one or more acoustic transducers 108 can be coupled to substrate 105. The one or more acoustic transducers 108 can be configured to provide acoustic waves to the fluid 106 in the fluid-retaining region 107 such that the fluid 106 is rotated. In some examples, the one or more acoustic transducers 108 can include one or more single-phased focused transducers (SPFT). In some examples, the one or more acoustic transducers 108 can include a pair of acoustic transducers. In some examples, the one or more acoustic transducers 108 can include two pairs of acoustic transducers. In some examples, the two pairs of acoustic transducers can be opposed pairs of acoustic transducers, as illustrated, for example, in FIG. 3 (e.g., the opposed pairs of acoustic transducers can be positioned on opposing sides of the fluid-retaining region 107). The one or more acoustic transducers 108 can be offset from a center of the fluid 106, such that the fluid 106 is rotated by the acoustic wavers provided to the fluid 106 in the fluid-retaining region 107. In some examples, the one or more acoustic transducers 108 provide a circulatory acoustic streaming effect to the fluid 106 in the fluid-retaining region 107. The one or more acoustic transducers 108 can be configured to rotate the fluid 106 in a stable spinning state. For example, the one or more acoustic transducers 108 can be configured to rotate the fluid 106 in a whirlpool-like manner. For example, the one or more acoustic transducers 108 can be configured to provide acoustic waves to the edges of the fluid 106 such that the fluid 106 is rotated.

The system 100 can include a disc 102. In some examples, the disc 102 can include one or more microfluidic channels 104(a), 104(b), 104(c). The one or more microfluidic channels 104(a), 104(b), 104(c) can be configured to receive a liquid sample. In some examples, the microfluidic channels 104(a), 104(b), 104(c) can be engraved in the surface of the disc 102. The disc 102 can be operable to be disposed on top of the fluid 106 in the fluid-retaining region 107. The disc 102 can be operable to rotate with the fluid 106 in the fluid-retaining region 107. For example, as the fluid 106 is rotated by the one or more acoustic transducers 108, the disc 102 can rotate with the fluid 106. For example, rotation line 110 illustrates the rotation of the fluid 106 in the fluid-retaining region 107, which thereby rotates the disc 102.

The rotation of the disc 102 can establish a centrifugal field and an induced fluid flow within the one or more microfluidic channels 104(a), 104(b), 104(c). The centrifugal field and induced fluid flow can be operable to transport particles within the liquid sample along the one or more microfluidic channels 104(a), 104(b), 104(c) at velocities dependent on one or more of particle size and particle density, thereby effecting separation and/or enrichment of a selected particle fraction of the liquid sample. In some examples, the selected particle fraction can include exosomes. In some examples, given the greater size and particle density of exosomes as compared to other particles in the liquid sample, the exosomes traverse the one or more microfluidic channels 104(a), 104(b), 104(c) at a greater velocity than the remaining particles in the liquid sample. In this manner, larger particles (i.e., exosomes) are separated and enriched near a distal end (e.g., near the perimeter of the disc 102) of the one or more microfluidic channels 104(a), 104(b), 104(c). These enriched and separated particles can be used to diagnose diseases and disorders. For example, the enriched and separated particles can be used to diagnose cancers and other diseases and disorders having biomarkers contained in exosomes. While the separation and enrichment of the selected particle fraction is described with exosomes as an example, it will be appreciated that other selected particle fractions can be separated (e.g., particles with decreased size and/or density can be found towards the center of the disc 102 and can be used for various diagnostic applications). In some examples, large particles can be separated and enriched, then subsequently removed. The disc 102 can then be rotated again to separate and enrich a smaller selected particle fraction. This process can be repeated until a desired selected particle fraction is obtained.

In some examples, the system 100 can include a power supply. The power supply can be operable to provide power to the one or more acoustic transducers 108 such that the acoustic transducers 108 can provide acoustic waves to the fluid-retaining region 107.

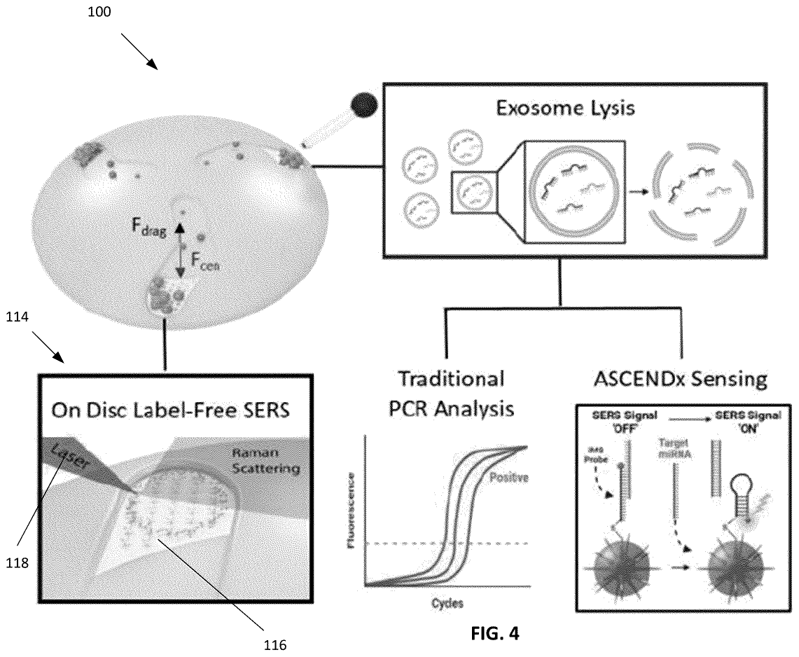

As illustrated in FIG. 4, the system 100 can further include at least one biosensing unit 114. The at least one biosensing unit 114 can be located along at least one of the one or more microfluidic channels 104(a), 104(b), 104(c). In some examples, the at least one biosensing unit 114 can be located at the distal end of each of the one or more microfluidic channels 104(a), 104(b), 104(c). The at least one biosensing unit 114 can be configured to determine a composition of the liquid sample at the location in which the biosensing unit 114 is located along the one or more microfluidic channels 104(a), 104(b), 104(c). In some examples, the at least one biosensing unit 114 can be located at the distal end of each of the one or more microfluidic channels 104(a), 104(b), 104(c) such that the at least one biosensing unit 114 can be configured to determine the composition of the separated and enriched selected particles of the liquid sample after the disc 102 is rotated. In some examples, the at least one biosensing unit 114 can include a plurality of biosensing units located at different locations along the one or more microfluidic channels 104(a), 104(b), 104(c). For example, the plurality of biosensing units can be configured to determine the composition of a selected particle fraction of the liquid sample at various locations along the one or more microfluidic channels 104(a), 104(b), 104(c) after separation and enrichment of the selected particle fraction of the liquid sample (e.g., after spinning of the disc 102).

In some examples, the at least one biosensing unit 114 can include a label-free surface enhanced Raman scattering sensor. For example, the at least one biosensing unit 114 can include a surface-enhanced Raman spectroscopy region 116. The surface-enhanced Raman spectroscopy region 116 can include a plurality of plasmonic nanostructures immobilized at a location along at least one of the plurality of microfluidic channels 104(a), 104(b), 104(c). In some examples, the plurality of plasmonic nanostructures can be configured to provide electromagnetic field enhancement sufficient to allow detection target-induced spectral signatures with a Raman spectrometer. For example, the plurality of plasmonic nanostructures can be configured to concentrate light to significantly amplify the weak Raman signal of nearby molecules (e.g., exosomes or other selected particle fraction that has been separated and enriched in the surface-enhanced Raman spectroscopy region), thereby enabling detection and analysis of the nearby molecules.

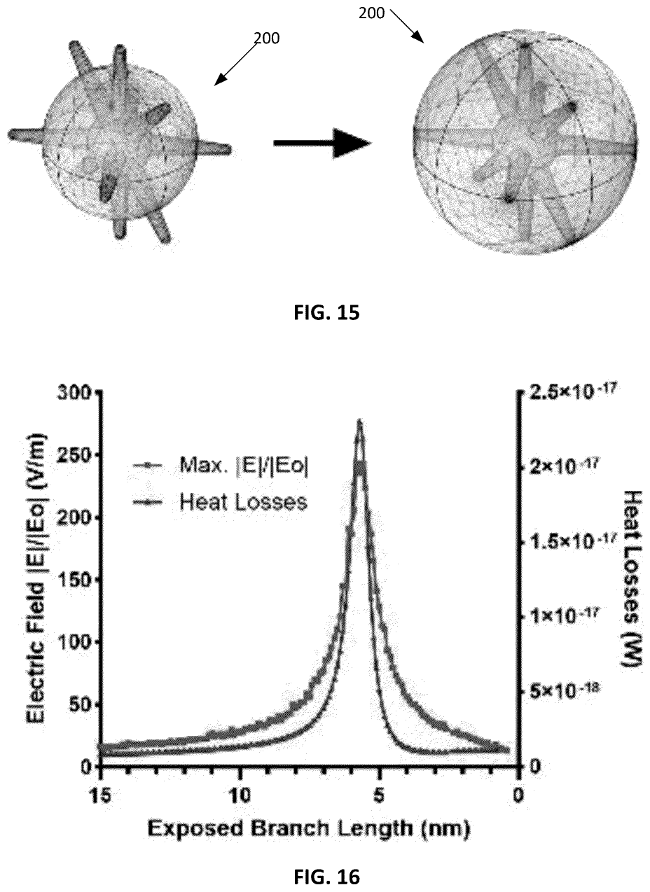

In some examples, the plurality of plasmonic nanostructures can include bimetallic nanostars having a gold core and a silver shell with a shell thickness configured to maximize the electromagnetic field enhancement. For example, FIG. 15 illustrates a bimetallic nanostar 200 in accordance with the present disclosure. The bimetallic nanostar 200 is further described herein.

The at least one biosensing unit 114 can further include an excitation light source 118. The excitation light source 118 can be configured to illuminate the surface-enhanced Raman spectroscopy region at an excitation wavelength sufficient to induce a reporter Raman signal. The reporter Raman signal can be indicative of the composition of the liquid sample in the surface-enhanced Raman spectroscopy region 116. The biosensing unit 114 can include a Raman spectrometer operable to detect the reporter Raman signal, thereby detecting the composition of the liquid sample in the surface-enhanced Raman spectroscopy region 116

In some examples, the excitation light source 118 can include a laser. In some examples, the excitation light source 118 can provide light at a desired wavelength for inducing the reporter Raman signal. For example, the wavelength of the excitation light can be about 600 nanometers (nm) to about 700 nm. In some examples, the excitation light can be about 630 nm to about 640 nm. In some examples, the excitation light can be about 633 nm.

In some examples, the at least one biosensing unit 114 can further include a plurality of probes covalently attached to the plurality of plasmonic nanostructures. The plurality of probes can be configured as inverse molecular sentinel probes, that, upon hybridization with one or more biomarkers in the liquid sample, undergo a conformational change that increases the reporter Raman signal. The increased reporter Raman signal, detected by the Raman spectrometer, can be indicative of the presence of one or more target biomarkers in the liquid sample. Since the selected particle fraction of the liquid sample is separated and enriched in the surface-enhanced Raman spectroscopy region 116, detection of the increased reported Raman signal is significantly more efficient than in non-enriched liquid samples.

In some examples, the plurality of probes can include a plurality of nucleic acid or nucleic acid analog probes. In some examples, the plurality of nucleic acid or nucleic acid analog probes can include multiplexed inverse molecular sentinel probes specific for at least one microRNA (miRNA) target.

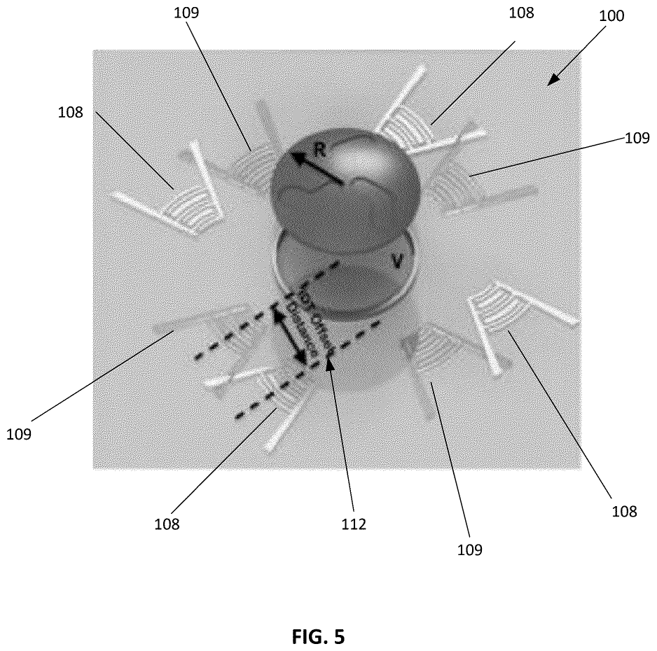

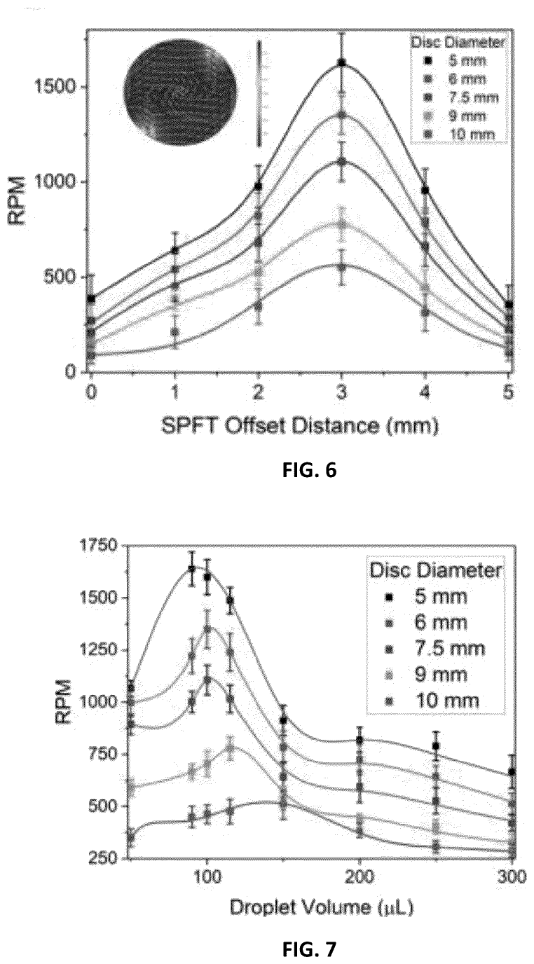

As illustrated in FIG. 5, the one or more acoustic transducers 108 can be offset from the center of the fluid 106 in the fluid-retaining region 107. For example, the one or more acoustic transducers can be offset from the center of the fluid 106 by an offset distance 112. The offset distance 112 can be defined as a distance of a transducer from a location where the transducer would provide acoustic waves directly to the center of the fluid 106 in the fluid-retaining region 107. The offset distance 112 moves the one or more acoustic transducers 108 to be offset from the center of the fluid 106 in the fluid-retaining region 107 without changing the direction in which the acoustic waves are transmitted from the one or more acoustic transducers 108. In some examples, the offset distance 112 can be selected such that the rotation speed of the fluid 106, and thereby the rotation speed of the disc 102, is maximized. For example, if the fluid 106 has a radius of about 5 millimeters (mm) the offset distance 112 can be about 3 mm. FIG. 6 illustrates the offset distance of the one or more transducers 108 and corresponding increase in rotation speed of the fluid 106.

In some examples, the disc 102 can be open to the external environment. In other examples, the system 100 can include a membrane. The membrane can be configured to enclose the one or more microfluidic channels 104(a), 104(b), 104(c). For example, after the liquid sample is placed in the one or more microfluidic channels 104(a), 104(b), 104(c), the membrane can be coupled to the disc 102, thereby enclosing the liquid sample within the one or more microfluidic channels 104(a), 104(b), 104(c). In some examples, the membrane can be operable to reduce and/or prevent sample evaporation and ambient contamination.

In some examples, the system 100 can further include a temperature control element. For example, the temperature control element can be placed under the substrate 105. In some examples, the temperature control element can cool the substrate 105, and thereby the remaining components of the system 100. In some examples, the temperature control element is operable to reduce heating and maintain the integrity of the liquid sample. In some examples, the temperature control element can include a Peltier cooling plate.

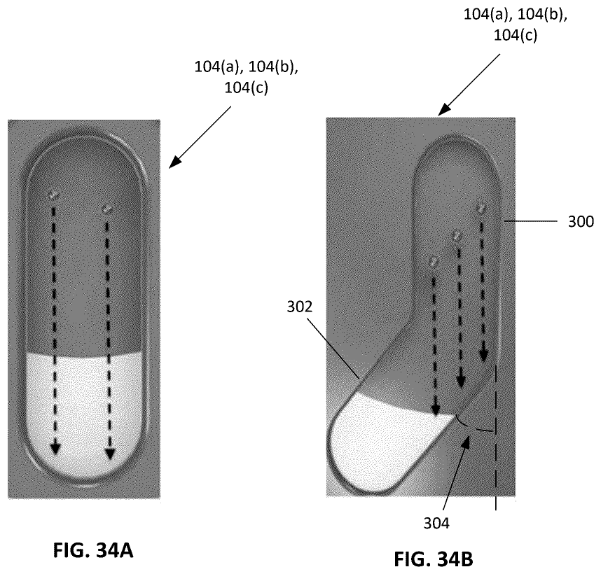

In some examples, the one or more microfluidic channels 104(a), 104(b), 104(c) can have geometry. FIGS. 34A-34B illustrate various geometries of the microfluidic channels 104(a), 104(b), 104(c). As illustrated in FIG. 34A, the one or more microfluidic channels 104(a), 104(b), 104(c) can have a straight geometry. As illustrated in FIG. 34B, the one or microfluidic channels 104(a), 104(b), 104(c) can have a slanted (e.g., tilted) geometry. For example, as illustrated in FIG. 34B, the one or more microfluidic channels 104(a), 104(b), 104(c) can have a first portion 300 and a second portion 302. The first portion 300 can transition to the second portion 302 at an angle 304. The second portion 302 can extend from the first portion 300 at the angle 304. In some examples, the angle 304 can be measured as a degree in which the second portion 302 shifts from the first portion 300. The slanted geometry of the microfluidic channels 104(a), 104(b), 104(c) can be operable to increase the efficiency of separation and/or enrichment of the selected particle fraction. For example, the slanted geometry can shorten a transport path of the selected particles, thereby allowing the particles to be separated faster.

In some examples, the angle 304 can be about 1 degree to about 90 degrees. In some examples, the angle 304 can be less than 90 degrees. In some examples, the angle 304 can be about 1 degree to about 5 degrees, about 5 degrees to about 10 degrees, about 10 degrees to about 15 degrees, about 15 degrees to about 20 degrees, about 20 degrees to about 25 degrees, about 25 degrees to about 30 degrees, about 30 degrees to about 35 degrees, about 35 degrees to about 40 degrees, about 40 degrees to about 45 degrees, about 45 degrees to about 50 degrees, about 50 degrees to about 55 degrees, about 55 degrees to about 60 degrees, about 60 degrees to about 65 degrees, about 65 degrees to about 70 degrees, about 70 degrees to about 75 degrees, about 75 degrees to about 80 degrees, about 80 degrees to about 85 degrees, or about 85 degrees to about 90 degrees.

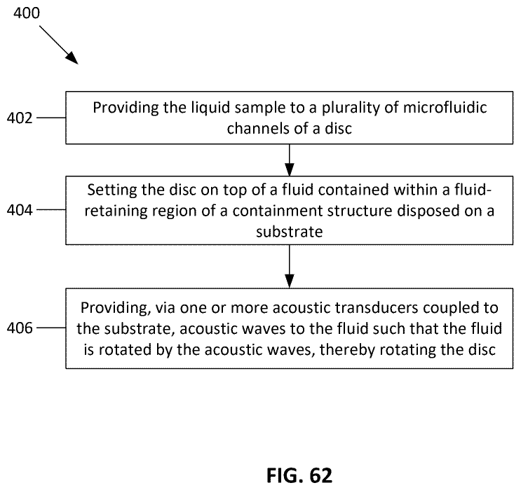

Further provided herein is a method for separation and/or enrichment of particles in a liquid sample. FIG. 62 illustrates an example of a method 400 for separation and/or enrichment of particles in a liquid sample. The method 400 can be conducted using the devices and systems described herein.

At block 402, the method 400 can begin by providing a liquid sample to a plurality of microfluidic channels of a disc. The liquid sample can include a patient sample for testing and diagnosis of various diseases and disorders. In some examples, the liquid sample can include blood, saliva, and/or other bodily fluids. While the microfluidic channels are described as a plurality of microfluidic channels, it will be appreciated that a single microfluidic channel can be used. In some examples, the liquid sample can be placed in the plurality of microfluidic channels via a syringe, pipette, or other device operable to hold a liquid sample.

At block 404, the method 400 can include setting the disc on top of a fluid contained within a fluid-retaining region of a containment structure disposed on a substrate. For example, the fluid can include a fluid with a low viscosity such that the fluid is easily rotatable by external forces (e.g., acoustic waves). In some examples, the fluid can include a water droplet. The disc can be operable to sit atop the fluid. In some examples, the disc can be operable to move in conjunction with the fluid (e.g., when the fluid rotates or moves the disc moves or rotates).

At block 406, the method 400 can include providing, via one or more acoustic transducers coupled to the substrate, acoustic waves to the fluid such that the fluid is rotated by the acoustic waves, thereby rotating the disc. For example, acoustic waves can be provided near the edge or edges of the fluid such that the acoustic waves induce a whirlpool-like rotation of the fluid. In some examples, the fluid-retaining region is operable to enclose the fluid such that the whirlpool-like rotation of the fluid can occur (e.g., the side walls of the containment structure defining the fluid-retaining region influence the fluid to rotate when the acoustic waves are provided).

In some examples, the rotation of the disc can be operable to establish a centrifugal field and an induced fluid flow within the plurality of microfluidic channels to transport particles within the liquid sample. The centrifugal field and induced fluid flow can cause the particles to be transported along the plurality of microfluidic channels at velocities dependent on one or more of particle size and particle density. The velocities of the particles can effect separation and/or enrichment of a selected particle fraction of the liquid sample. For example, particles with greater size and/or density can have a higher velocity than smaller particles. Particles with greater size and/or density are separated and enriched at a distal end (e.g., near an edge or perimeter of the disc) of the plurality of microfluidic channels. For example, exosomes in bodily fluids are typically larger and have greater densities than other components in the bodily fluids. By spinning the disc, exosomes can be separated and enriched at the distal end of the microfluidic channels. The exosomes can then be used in diagnostic methods to diagnose diseases and disorders.

In some examples, the method 400 can further include providing, via an excitation light source, an excitation light to a surface-enhanced Raman spectroscopy region along at least one of the plurality of microfluidic channels to induce a reporter Raman signal. In some examples, the reporter Raman signal can be indicative of a composition of the liquid sample in the surface-enhanced Raman spectroscopy region. For example, in the case of exosome separation and enrichment, the surface-enhanced Raman spectroscopy region can be at or near the distal end of the plurality of microfluidic channels. In this manner, the separated and enriched exosomes can be detected.

In some examples, the surface-enhanced Raman spectroscopy region can include a plurality of plasmonic nanostructures. The plurality of plasmonic nanostructures can be configured to provide electromagnetic field enhancement sufficient to detect target-induced spectral signatures.

In some examples, the method 400 can further include detecting an increase to the reporter Raman signal when a plurality of nucleic acid or nucleic acid analog probes covalently attached to the plurality of plasmonic nanostructures hybridize with one or more biomarkers in the liquid sample and undergo a conformational change. In some examples, the hybridization of the plurality of nucleic acid or nucleic acid analog probes with the one or more biomarkers in the liquid sample, detected by the increased Raman reporter signal, can be indicative of various disease and/or disorder states. In some examples, the one or more biomarkers can include one or more miRNA targets.

In some examples, the plurality of nucleic acid or nucleic acid analog probes can include multiplexed inverse molecular sentinel probes specific for at least one miRNA target. In some examples, the plurality of nucleic acid or nucleic acid analog probes can include oligonucleotide probes and the at least one miRNA target can include miR-21 and/or miR-221. For example, miR-21 and/or miR-221 can be indicative of circulating colorectal cancer, thereby allowing the method 400 to diagnose circulating colorectal cancer.

It will be appreciated that the nucleic acid or nucleic acid analog probes described herein are examples only and other nucleic acid or nucleic acid analog probes can be used to target other miRNA targets. It will be appreciated that the use of nucleic acid or nucleic acid analog probes are simply an example of probes that may be used. Other types of probes that hybridize with other types of biomarkers can be used to detect different diseases and/or disorders.

In some examples, the method 400 can further include providing, via a temperature control element thermally coupled to the substrate, temperature regulation to maintain a sample temperature within a predetermined range. For example, the temperature control element can provide cooling to the substrate as the acoustic waves propagate through the fluid to rotate the fluid and the disc. The cooling can be operable to keep the temperature of the liquid sample in the plurality of microfluidic channels within a predetermined range. In some examples, the predetermined range can be about ±5 degrees Celsius of room temperature. In some examples, the predetermined range can be about ±1 degree Celsius of room temperature. In this manner, spoiling of the liquid sample can be reduced and/or prevented.

In some examples, the method 400 can further include treating the detected disease or disorder. For example, the detected disease or disorder can be treated using methods known in the art.

### EXAMPLES

### Example 1

The examples provided herein provide a setup of the system 100 in accordance with various embodiments.

### Introduction

Exosomes are cell-derived nanovesicles that have recently gained popularity as potential biomarkers for liquid biopsies due to their plethora of molecular cargo, such as nucleic acids and proteins. As a result, circulating exosomes are considered promising non-invasive biomarkers for investigating disease development, progression, and early disease detection. Exosome isolation, however, is challenging due to their small size and cohabitation with numerous other bioparticles in bodily fluids. Although several methods of separating exosomes have been developed, such as ultracentrifugation, polymer-assisted precipitation, and ultrafiltration, current techniques fail to meet research and clinical needs due to a lengthy processing time, large sample consumption, and limited purity and yield.

Clinical applications of exosomes are further hindered by limitations in biomarker analysis. Their lengthy procedures and cost-prohibitive reagent and instrumentation requirements limit conventional detection methods such as digital droplet polymerase chain reaction (PCR), benchtop RT-qPCR, sequencing, and microarray analysis. To circumvent these drawbacks, other sensing strategies have been implemented, including isothermal amplification, lateral flow tests, and electrochemical sensing assays, but they lack the specificity and sensitivity needed for widespread adoption in clinical settings due to the low concentrations of biomarkers in bodily fluids. Therefore, biomarker detection remains challenging without a unified exosome enrichment and in-situ analysis platform to purify and concentrate biomarkers before detection.

Recently, microfluidics has emerged as a promising candidate for exosome separation and analysis. Current microfluidic technologies employ a variety of mechanisms such as electric fields, magnetics, and acoustics to control bioparticles and actuate fluids. However, existing microfluidic separation technologies have difficulties manipulating particles below 50 nm in size and removing the small, non-exosomal contaminants such as lipoproteins, leading to less reliable and consistent biomarker diagnosis. Additionally, current technologies often produce relatively dilute samples and need help concentrating the exosomes, limiting the molecular information available for analysis. Furthermore, many existing microfluidic systems must be coupled with bulky analytical systems, making them unsuitable for point-of-care testing. For example, microfluidic separation often uses counting approaches such as nanoparticle tracking analysis and dynamic light scattering. Still, these technologies have bulky fingerprints and provide little diagnostic information. Conventional molecular assays such as western blots and ELISA require large sample volumes, hampering their application in settings with large patient cohorts or limited specimen availability. Hence, there exists a need for rapid enrichment technology with integrated multiplexed biomarker sensing to bring the next-generation liquid biopsy analysis into fruition.

The system 100 described herein overcomes these limitations. The system 100 allows the enrichment and detection of exosomes using a rotating microfluidic disc 102 integrated with a multiplexed, plasmonic nanostar-based miRNA assay. Acoustofluidic disc rotation is generated by coupling surface acoustic waves (SAWs) and the fluid 106 on which the disc 102 floats to enable centrifugation and fluid actuation within the microfluidic channels 104(a), 104(b), 104(c) on the surface of the disc. Various functionalities are demonstrated, such as the separation and concentration of both micro and nanoscale objects. The interplay between the centrifugal and drag forces leads to size and density-dependent transport velocity, which is utilized to separate the relatively large exosomes from protein contaminants in plasma. Additionally, a bimetallic nanostar substrate is functionalized on the surface of the disc 102 to verify the enrichment mechanism through label-free Surface-enhanced Raman scattering (SERS) sensing of the concentrated exosome sample. Furthermore, diagnostic potential of the system 100 is demonstrated through traditional RT-PCR and a multiplexed amplification-free SERS assay for detecting circulating colorectal cancer (CRC) miRNA biomarkers from patient plasma samples with high selectivity (95.8%) and specificity (100%).

### Results

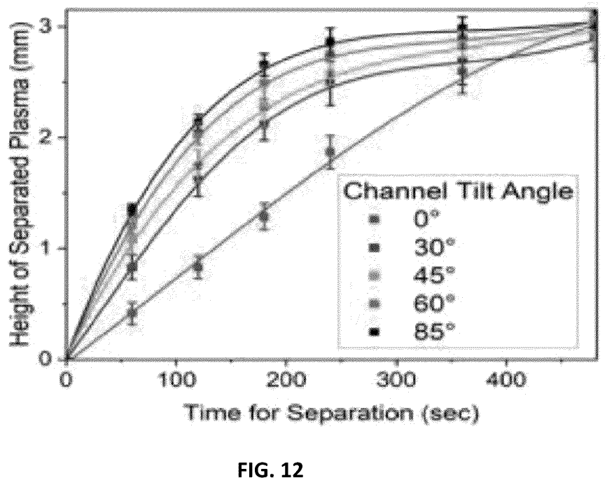

As shown in FIGS. 1-4, the system 100 consists of a droplet (e.g., fluid 106) confined to a polydimethylsiloxane (PDMS) ring (e.g., containment structure 103) and two pairs of single-phased focused transducers (SPFT) (e.g., one or more transducers 108) aligned along the flanks of the droplet (e.g., fluid 106). A disc 102 with slanted channels (e.g., one or more microfluidic channels 104(a), 104(b), 104(c)) engraved on its surface sits atop the droplet (e.g., fluid 106). Four SAWs propagate into the droplet when an electrical signal is applied to the SPFTs, forming a circulatory acoustic streaming effect. As the input voltage increases, the droplet reaches a stable spinning state. The rotating droplet-disc system acts as a centrifuge when the acoustofluidic disc is placed on the droplet. As FIG. 4 illustrates, particles in the disc channels drift toward the disc edge, with larger particles migrating first. This phenomenon concentrates the large particles and creates a mechanism for separating the larger particles from the smaller ones that remain in the channel. The disc features a tilted channel geometry to enhance the enrichment further by shortening the path traveled by particles in the solution. FIG. 12 illustrates plasma separation heights per time for various channel tilt angles, where height is measured as distance from a proximal end (e.g., channel side near the center of the disc).

Differences in mass density cause phase separation between suspended particles in solution. This sedimentation of particles can be accelerated by applying a centrifugal force given by Eq. (1):

$${F}_{c} = {r}_{avg} {ω}^{2} {V}_{p} Δρ \qquad ( 1 )$$

Where r_{avg} is the average radial distance of particles from the center of rotation, Vₚ is the volume of the particle, ω is the angular velocity, and Δρ is the difference between the density of the particle and the density of the medium. As particles, rₚ migrate within a fluid of viscosity η, the particles experience a counteracting drag force proportional to particle velocity, v, given by Eq. (2):

$${F}_{drag} = 6 πη {r}_{avg} v \qquad ( 2 )$$

The interplay of the centrifugal force and the Stokes drag force determines the time t_{d} required for complete sedimentation of particles of diameter d across a channel width L, given by Eq. (3):

$${t}_{d} = \frac{18 η {r}_{avg} v}{{ω}^{2} {d}^{2} Δ {pr}_{avg}} \qquad ( 3 )$$

The commonly accepted vesicle density for exosomes is 1.19 g/cm³ with a medium viscosity of 1.55 cp. Using these values, a 5 mm diameter disc spinning at 4,000 rpm with 100 μm wide channels will pellet 100% of 100 nm exosomes in just 31 minutes.

The shape and composition of plasmonic nanomaterials have been widely investigated for use in SERS sensing applications. The optical properties of these materials are attractive for integration into disease biomarker assays. To monitor exosome separation, the channel end of each acoustofluidic spinning disc has been functionalized with a plasmonic biosensing unit composed of bimetallic plasmonic nanostars with morphology optimized to generate a strong local electromagnetic field enhancement via localized surface plasmon resonance at 633 nm. This local electric field enhancement can be described where E₀ is the magnitude of the incident electric field, g is the field enhancement averaged over the surface of the particle, and E(ω) is the average magnitude of the electric field radiated by the particle at the incident frequency. E(ω) can be given by Eq. 4:

$$E ( ω ) = {gE}_{0} \qquad ( 4 )$$

At a Stokes-shifted wavelength, there is a corresponding electric field enhancement factor E(ω′); however, the approximation E(ω)=E(ω′) can be made, since the plasmon width is relatively large due to the Stokes-shift. With the electric field enhancement factor, the SERS electromagnetic enhancement factor EF, which describes the intensity ratio between SERS and Raman scattering for a given molecule, is given by Eq. 5:

$$EF = {\vert E ( ω ) \vert}^{4} \qquad ( 5 )$$

By leveraging the local electromagnetic field enhancement generated by the plasmonic nanostar surface, SERS-based label-free exosome enrichment verification method on-disc can be established, depicted in the lower left of FIG. 4.

The inverse Molecular Sentinel (IMS) assay can be integrated within the plasmonic biosensing unit of the system 100 to detect CRC miRNA biomarkers contained within enriched exosome aliquots derived from patient plasma. The amplification-free assay leverages a nonenzymatic DNA strand displacement process to trigger a conformational change of the probe and subsequently increase the SERS signal in the presence of target miRNA. Shown in the bottom right of FIG. 4, the iMS probe is functionalized to the plasmonic nanostar surface and hybridized with a partially complementary placeholder strand. This hybridization complex results in a negligible SERS signal due to the distance created between the Raman dye and the plasmonic nanostar surface. The placeholder strand is displaced from the miRNA probe when in the presence of target miRNA, forming an entirely complementary duplex. Now free, the iMS probe self-hybridizes to form a hairpin structure. This conformational change brings the Raman dye within the area of high electric field enhancement along the plasmonic nanostar surface, resulting in a strong SERS signal capable of providing diagnostic information from patient tissue and plasma samples.

### Characterization of the Acoustic Fluidic Disc Unit of the System

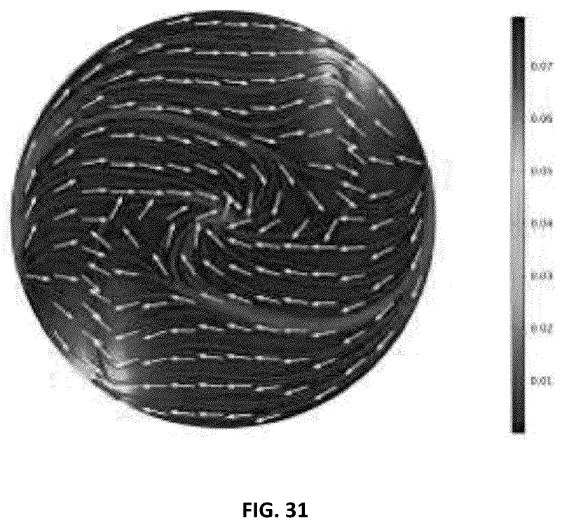

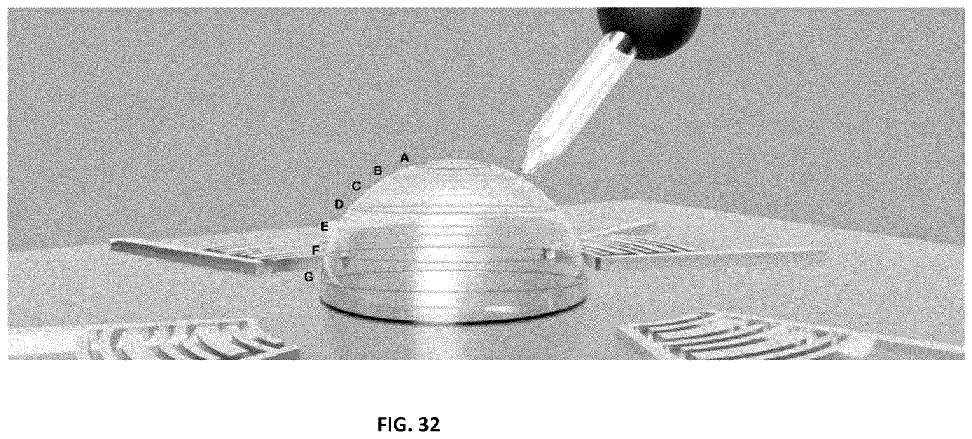

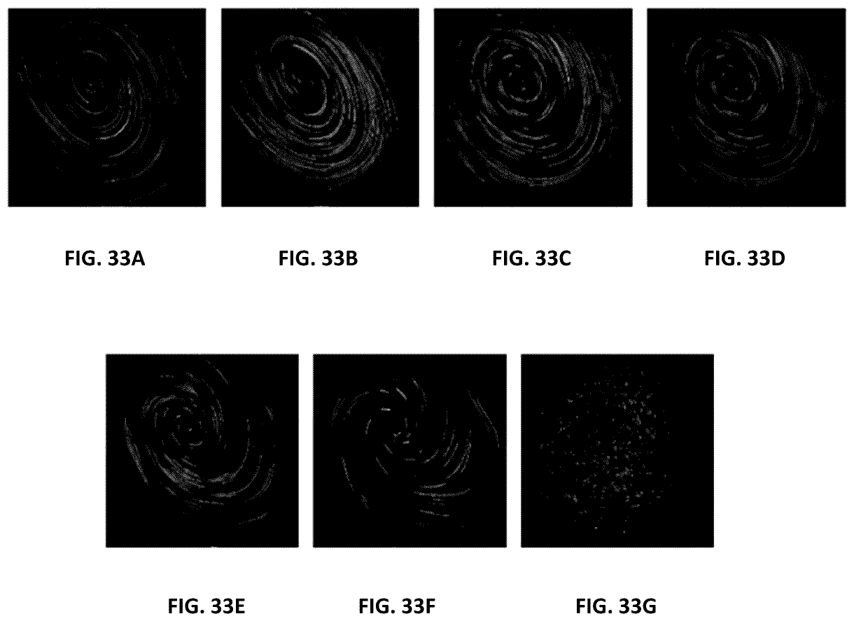

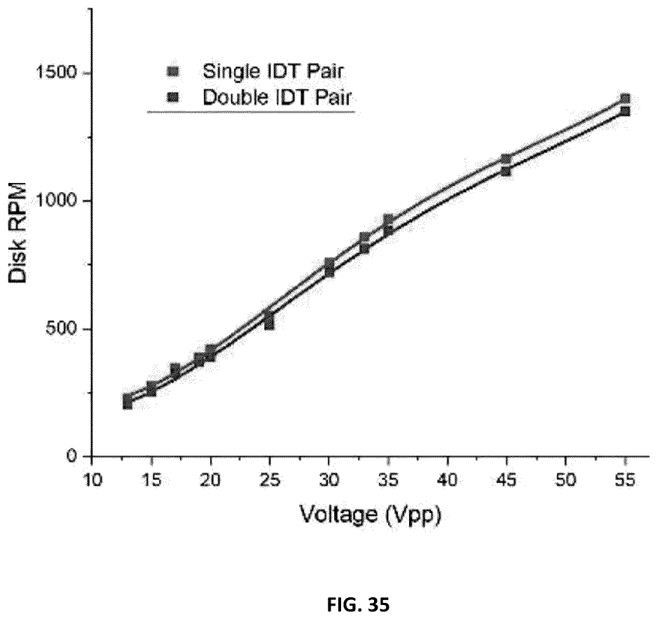

FIG. 32 illustrates an experimental acoustic streaming pattern within a droplet (e.g., fluid within the fluid-retaining region). FIGS. 33A-33G illustrate a series of images showcasing the streaming pattern at different layers of the droplet. As the particles spiral down to the bottom of the droplet, the streaming pattern acts as a whirlpool tat drives particles to aggregate at the center. As SAWs enter the liquid, internal vortex streaming induces droplet rotation in the acoustofluidic disc unit of system, as illustrated in FIGS. 33A-33G. At low acoustic excitation, the droplet remains in its equilibrium shape due to the insufficient radiation pressure. As the excitation voltage increases, the droplet gradually experiences small vibrations until reaching a threshold value at which a stable spinning state is established, characterized by droplet deformation into an ellipsoidal shape with periodic rotational boundary oscillations. The region where the acoustic beam enters the droplet can significantly impact the droplet's rotation speeds. To characterize and optimize the performance of the acoustofluidic disc unit of the system, the effects of SPFT offset distance on droplet rotation were determined. Numerical simulations were performed to visualize the acoustic streaming pattern within the droplet and identify the ideal location for acoustic wave propagation by applying an acoustic beam on two flanks of the droplet (FIG. 31). The simulation results indicate that increasing SPFT offset distance maximizes the rotation speed due to the increased torque applied to the droplet axis. To validate the simulation results, the rotation speed of the droplet-disc system was determined with variable transducer offsets. For a droplet with a radius of 5 mm, the optimal SPFT offset distance is 3 mm, due to the spread of the acoustic beam not considered in the simulations and the additional SPFT pair included in the experiments (FIG. 35).

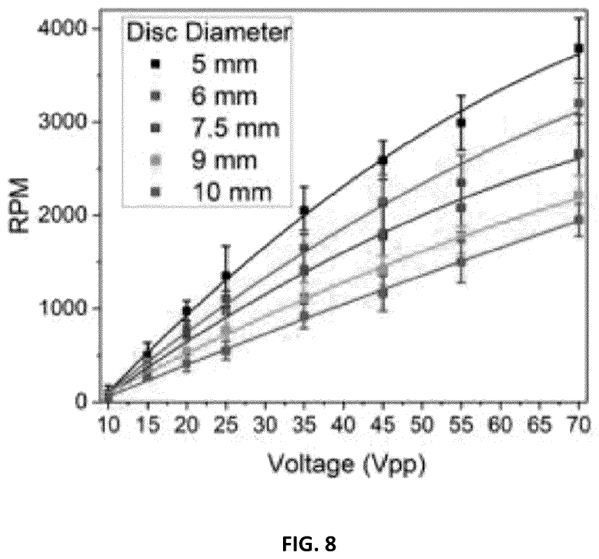

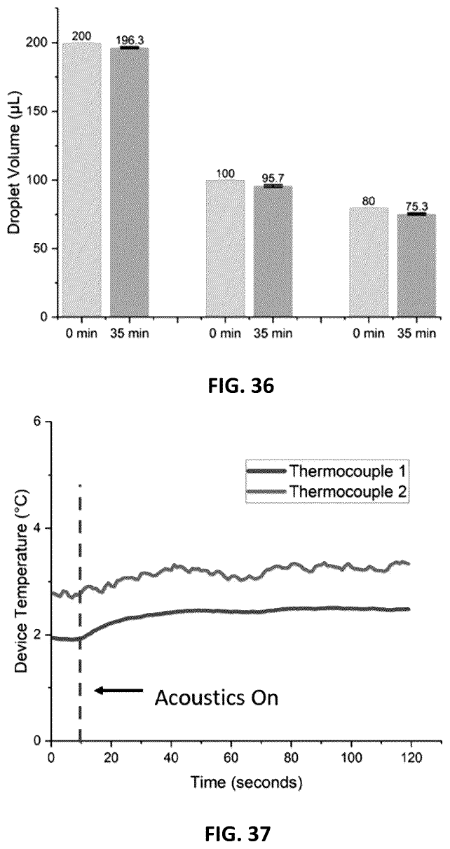

The effect of droplet volume on the rotation efficiency of the system was determined, as shown in FIG. 7. The results indicate that, generally, lower droplet volumes produce greater rotational speeds. However, the disc size plays an important role here, as sufficient liquid must be present to hold the disc and maintain droplet integrity. Additionally, the instability between the droplet and disc can induce pronounced precession of the disc, limiting the rotation speed. Therefore, there exists some optimal droplet volume corresponding to discs of various sizes to minimize precession and enable maximum centrifuge speeds. The viscosity of the liquid droplet is also an important consideration here. A water droplet was used, which experiences many reflections at the droplet interface. A more viscous liquid would attenuate the acoustic waves more and experience a lower streaming velocity. The relationship between input power and revolution speeds is shown in FIG. 8. As expected, higher applied voltage generates increased gyration. The droplets used experience negligible evaporation and thermal effects as voltage is increased due to their relatively large volume and enclosure by the PDMS ring and acoustofluidic disc, as illustrated in FIGS. 36-37.

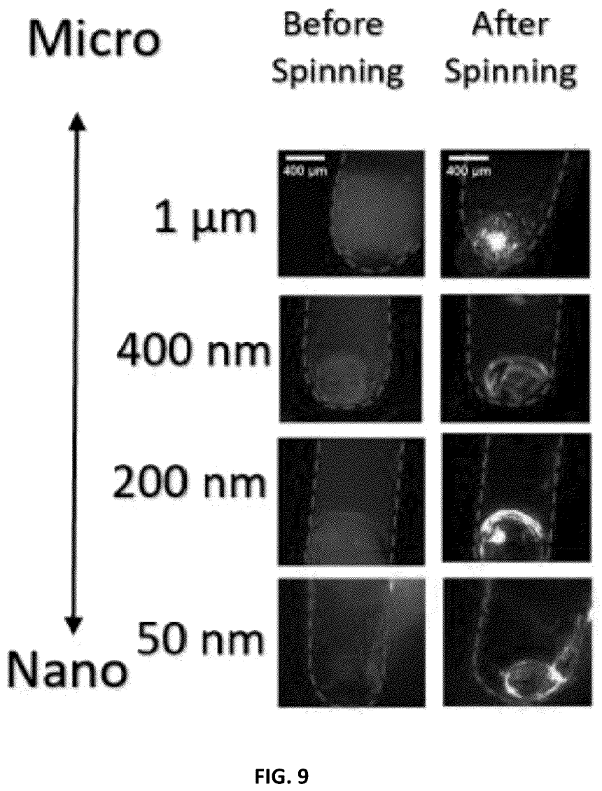

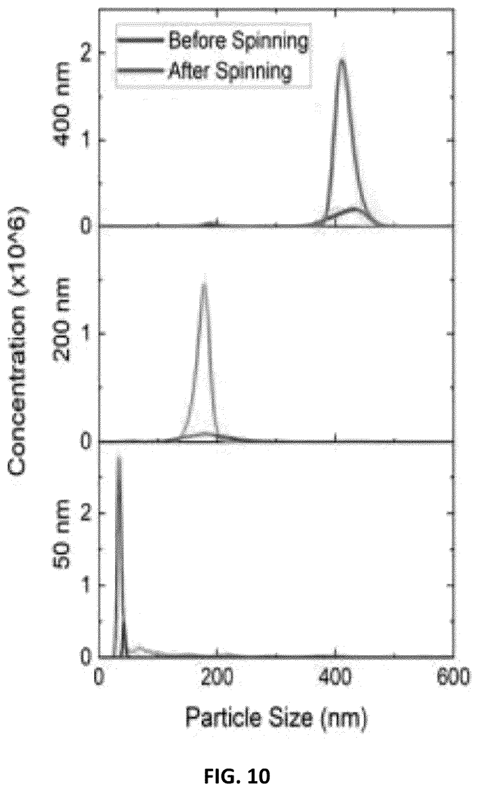

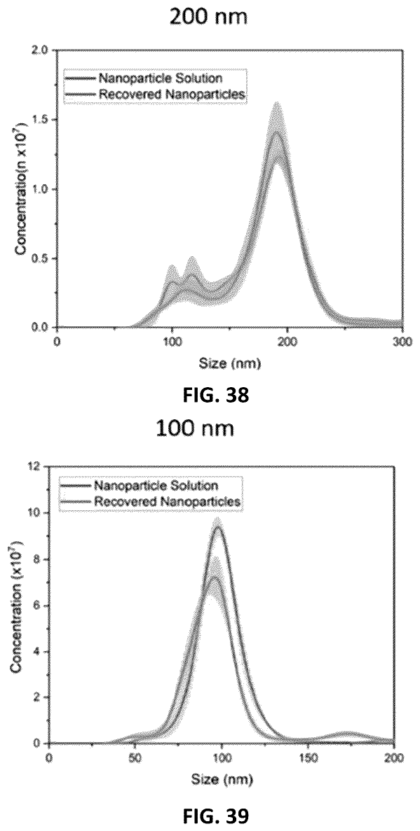

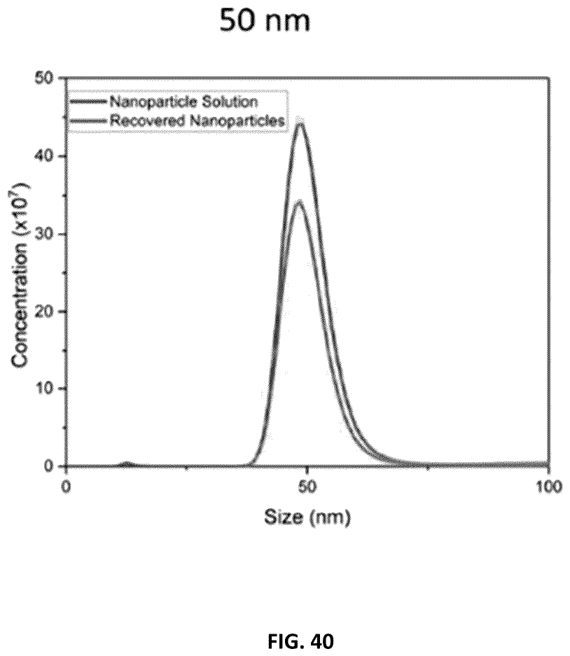

To showcase the enrichment capabilities of the system, fluorescent polystyrene beads were enclosed within the disc. As shown in FIGS. 9-10, the particles (50 nm, 200 nm, 400 nm, and 1 μm) are evenly dispersed throughout the channel before spinning but localized and heavily concentrated at the channel end after spinning, indicated by the significant changes in fluorescent intensity. The enrichment efficiency of the platform was quantified using NTA, shown in FIGS. 38-40, resulting in recovery rates of 94%, 85%, and 78% for 200 nm, 100 nm, and 50 nm particles, respectively. The results (FIG. 9) demonstrate that the system can concentrate samples from micro to nanoscales, making it ideally suited for a wide variety of applications in biology and medicine.

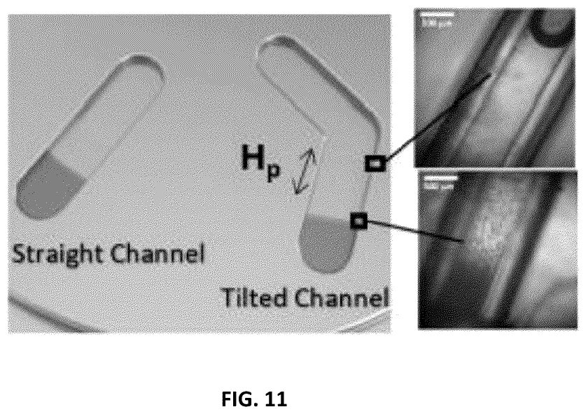

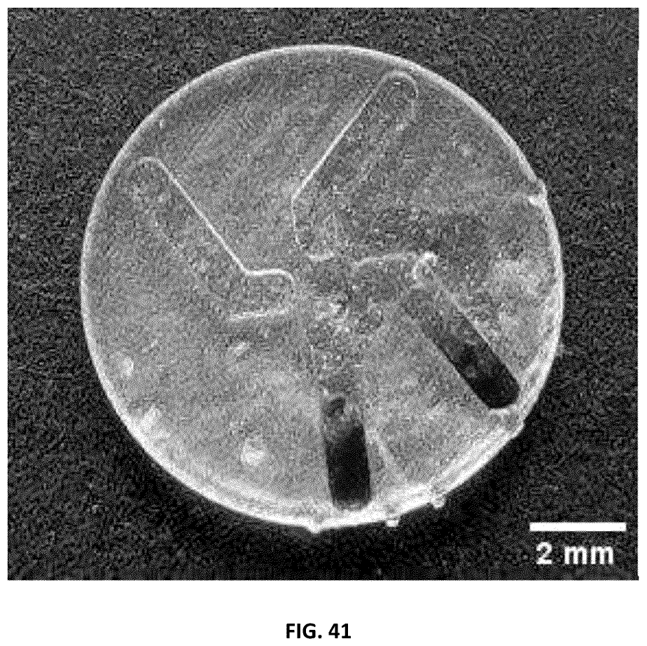

In addition to the concentration of homogenous solutions of particles, differential separation based on size was also evaluated. The dynamics between particle dimensions, density, and the centrifugal force generate distinct migration speeds for different-sized particles within the channel. To highlight the microscale separation potential of the system, the separation of plasma from whole blood is evaluated. 10 μL of blood was injected into the disc. As the disc spins, the larger blood cells experience greater centrifugal force, causing them to migrate to the channel end. As the height of the cell suspension decreases due to continued centrifugation, the height of the plasma layer Hp increases, creating a distinct interface between the cell suspension and plasma, as illustrated in FIGS. 11 and 41. The system can isolate highly pure plasma samples from whole blood, as shown in the rightmost inset images. The resulting serum can be easily retrieved with a pipette or syringe for additional processing and analysis downstream.

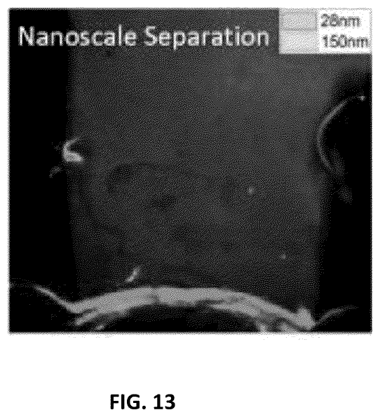

FIG. 13 presents the nanoscale separation of fluorescent particles. Here, both 28 nm and 150 nm particles were confined collectively in the channel. After spinning, the 150 nm particles relocate to the channel edge, while the 28 nm particles remain dispersed throughout the channel. Since the centrifugal force is proportional to the square of the particle size, the larger 150 nm particles experience significantly greater forces, causing them to concentrate well before the 28 nm particles, separating the two populations. Since exosomes range from 50-200 nm in size, this result highlights the potential for the system to separate exosomes from the many small (<10 nm) protein contaminants present in plasma.

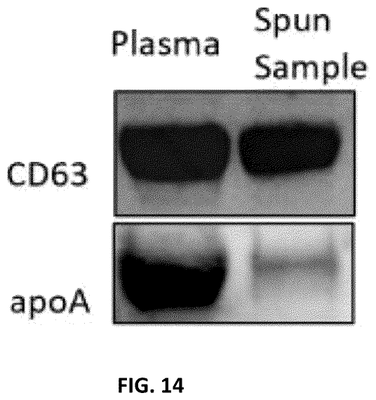

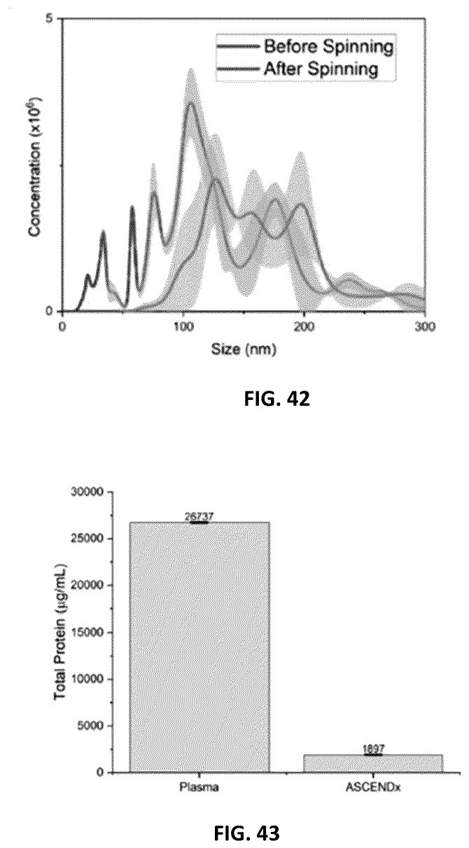

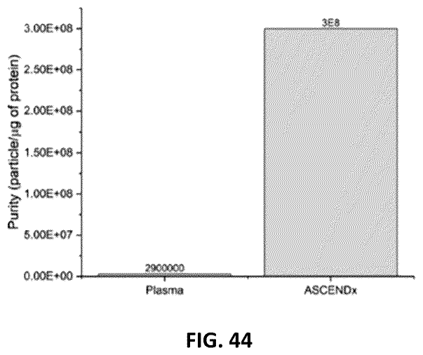

The separation of proteins from exosomes was also confirmed through western blot analysis. CD63 is a common surface protein found on exosomes, while apolipoprotein A is a small free protein contained in plasma but not in exosomes. As shown in FIG. 14, the original plasma sample contains high concentrations of both proteins, whereas the spun sample shows the presence of apolipoprotein A in lower concentrations while retaining the high CD63 content. Further validation of exosome yield and purity is demonstrated in FIGS. 42-44, which demonstrates a 120 times increase in the purity of acoustically enriched samples with a yield of 86%.

### Characterization of the Plasmonic Biosensing Unit

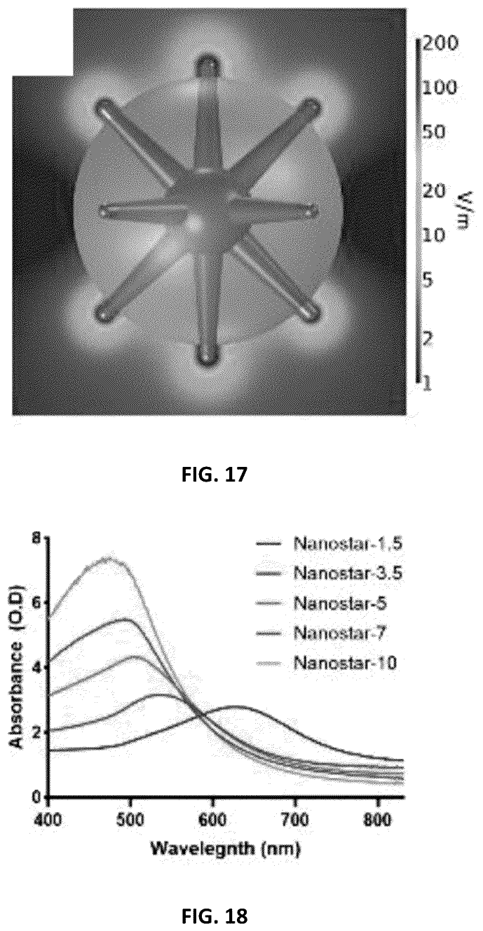

Plasmonic bimetallic nanostars are highly tunable sub-100 nm nanoparticles that produce intense local electromagnetic field enhancements to facilitate detection and sensing via SERS. Finite element modeling (FEM) of several bimetallic nanostar morphologies was performed with COMSOL Multiphysics 6.0 to determine the optimal nanostar morphology for disc integration. There, a gold nanostar model was constructed and held constant while the silver layer thickness was varied. FIG. 15 illustrates the range of silver thicknesses simulated in this study, 35 nm to 50 nm, or 15 nm to 0 nm exposed branch length. The highest normalized electric field enhancement factor and total heat losses for each simulated model are shown in FIG. 16. The heat loss values are obtained by integrating the resistive losses with the nanoparticle model volume and have been shown to be an accurate predictor of experimental SERS results. FIG. 17 depicts the nanostar morphology and corresponding normalized electric field with the greatest theoretical SERS performance, with a maximum electric field enhancement factor of 235.16 V/m and heat losses value of 2.3×10-17 W. This simulated nanostar had a silver layer thickness of 29.3 nm, or an exposed branch length of 5.7 nm. Overall, theoretical nanoparticle performance increases as the thickness of the silver layer increases until a maximum is reached, and then performance decreases as silver approaches the nanostar branch tips.

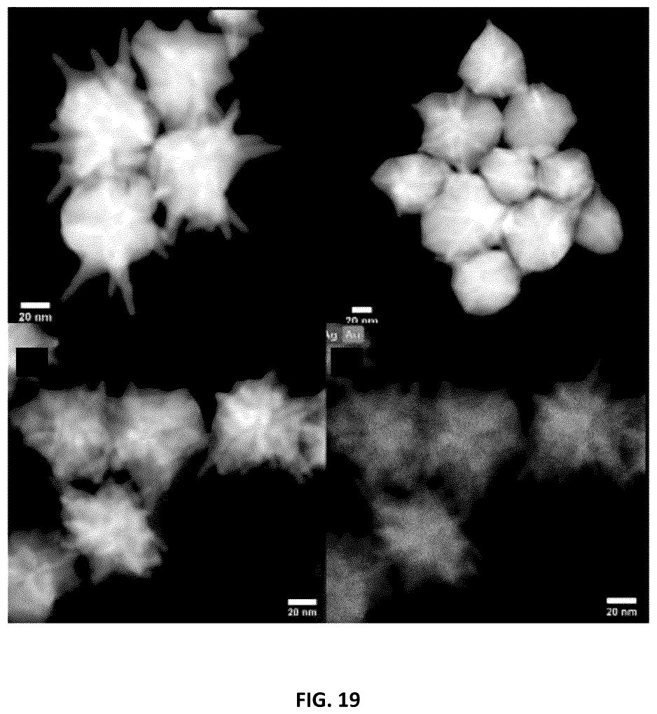

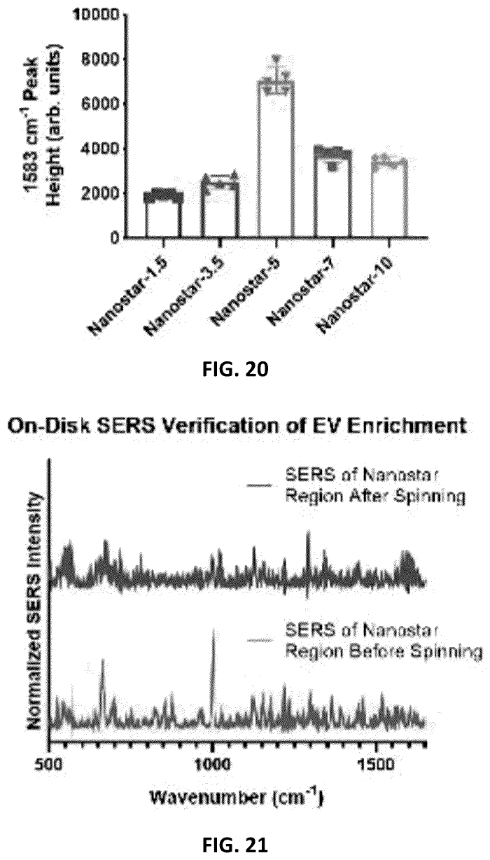

To verify the results of the FEM study, several different morphologies of bimetallic nanostars were synthesized. The formulation of the underlying gold nanostar was held constant while the thickness of the silver coating was varied. The resulting morphologies are referred to here as nanostar-1.5, 3.5, 5, 7, and 10, with the value denoting the mM concentration of silver added to coat the internal gold nanostars. The increase in silver thickness results in blue-shifting of the absorption spectra, as seen in FIG. 18. FIG. 19 contains High Angle Annular dark field (HAADF) scanning transmission electron microscopy (STEM) images of representative nanostar-1.5, 5, and 10 particles (e.g., top left is nanostar-1.5, top right is nanostar-5, bottom left is nanostar-10). Silver is deposited on the exposed core of the nanostar and grows outwards towards the branch tips. STEM-energy dispersive x-ray spectroscopy (EDS) was used to examine the elemental structure of the nanostar-5 particles, which clearly reveals the underlying gold nanostar encased in a silver layer. To test how these morphological changes would affect the local electric field enhancement and resulting SERS signal intensity, p-mercaptobenzoic acid was added to all 5 nanostar morphologies. The SERS spectra of all samples were recorded, and the intensity of each spectrum at 1583 cm-1 is shown in FIG. 20. As the silver layer thickness increases, the intensity of the SERS peak of interest increases, reaching a maximum with the nanostar-5 morphology, then decreasing as more silver is added to the particle surface, closely matching the results shown in FIG. 16.

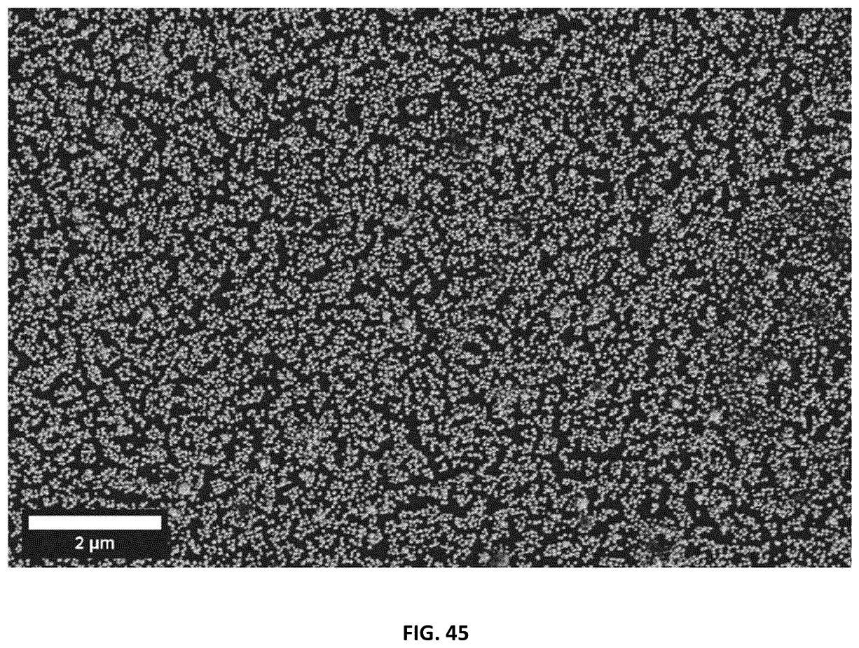

With the strong agreement between theoretical and experimental results, the nanostar-5 morphology was chosen as the nanoparticle type for incorporation onto the spinning acoustofluidic disc surface, as shown in the scanning electron microscopy (SEM) image in FIG. 45. The particles were incorporated into the end channel of the acoustofluidic disc to allow for on-disc label-free SERS verification of sample enrichment. FIG. 21 shows averaged SERS spectra obtained from the sensing portion of the disc with and without sample spinning. In the stationary sample, several peaks emerge, most notably at 1002 cm-1, which has been shown to dominate the SERS spectrum of albumin and other globular proteins and is attributed to the symmetric stretching mode of phenylalanine. This peak was noticeably reduced in the spun sample, as well as peaks at 665 cm-1, 875 cm-1, 850 cm-1, 1218 cm-1, and 1176 cm-1, which all are assigned to globular plasma protein. The reduction in the SERS signal of plasma protein after acoustofluidic disc-based sample concentration is evidence of extracellular vesicle enrichment. As larger extracellular vesicles are concentrated on the edges of the acoustofluidic disc, steric hindrance will decrease the amount of globular protein that can interact with the nanostar surface and generate a subsequent SERS signal.

### Validation of the System Through RT-PCR Clinical Samples

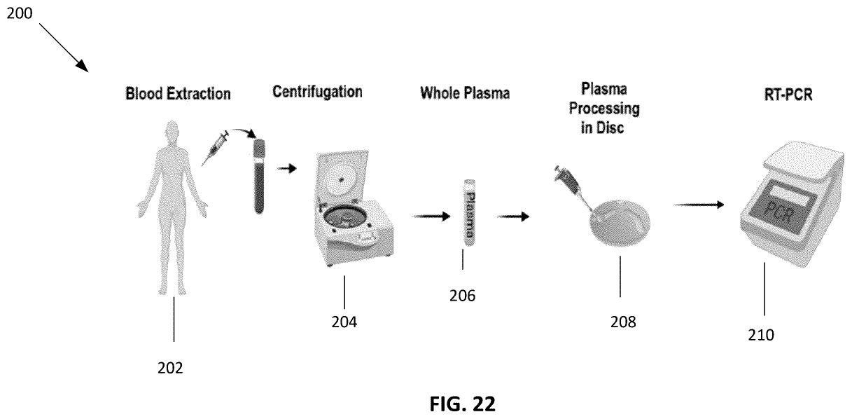

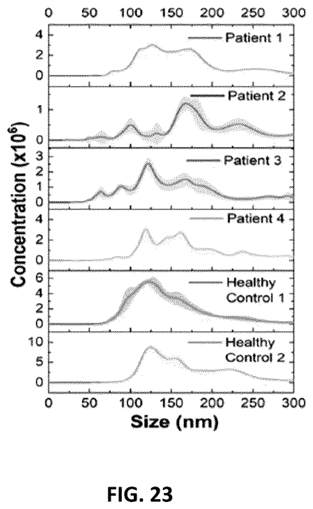

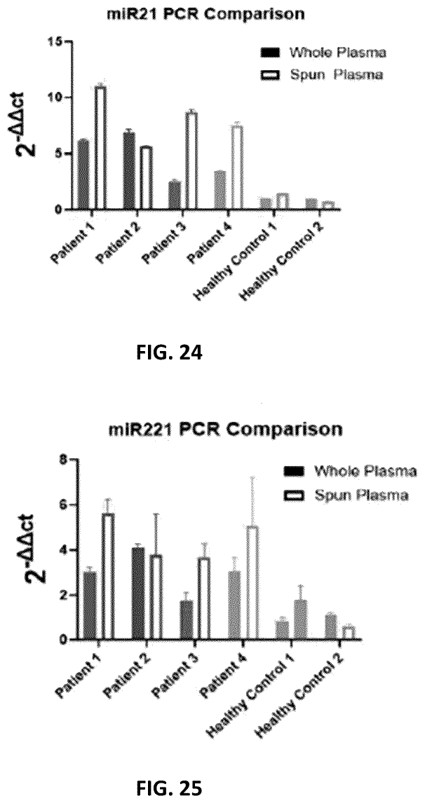

To highlight the diagnostic potential of the system through traditional RT-PCR analysis of CRC patient samples, 6 human plasma samples were analyzed (4 CRC patient samples and 2 healthy donors). The samples that were positive for CRC are denoted Patient 1, Patient 2, Patient 3, and Patient 4, while the control samples are denoted Healthy Control 1 and Healthy Control 2. FIG. 22 illustrates the entire sample-to-answer process, where patient blood samples were first extracted and conventionally centrifuged to isolate the plasma. This plasma sample is then loaded onto the acoutstofluidic disc, spun, and tested through RT-PCR. FIG. 23 shows the relative size distribution of each plasma sample after spinning in the system. The relative expression profiles of miR21 and miR221 were measured in each sample, as shown in FIGS. 24-25, showing high specificity between cancer patients and healthy individuals. Furthermore, to investigate the effects of the system on diagnostic outcomes, RT-PCR of unprocessed plasma and plasma processed through the system was conducted for each patient sample. For both CRC miRNA biomarkers, the results indicate that samples processed using the system have greater diagnostic utility than unprocessed plasma. Pearson correlation values between patients averaged 2^{-(ΔΔct)} values for each miRNA and diagnosis noticeably increased as a result of the system processing, from 0.764 to 0.900 in the case of miR-21 and from 0.802 to 0.902 in the case of miR-221. These findings agree with trends seen in the analysis of exosomes of colorectal cancer patients and further validate the utility that the system for exosome enrichment provides for improved biomarker detection.

### System: Sample to Answer

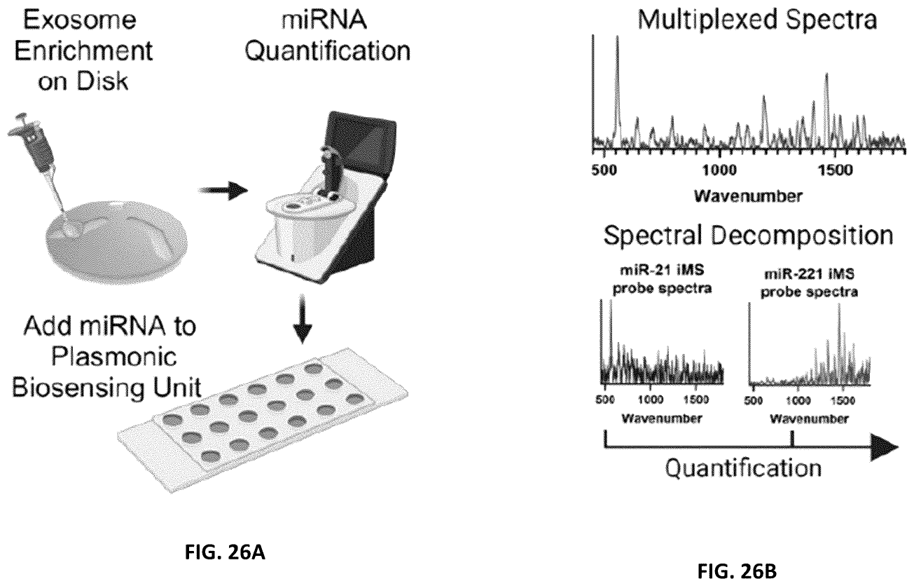

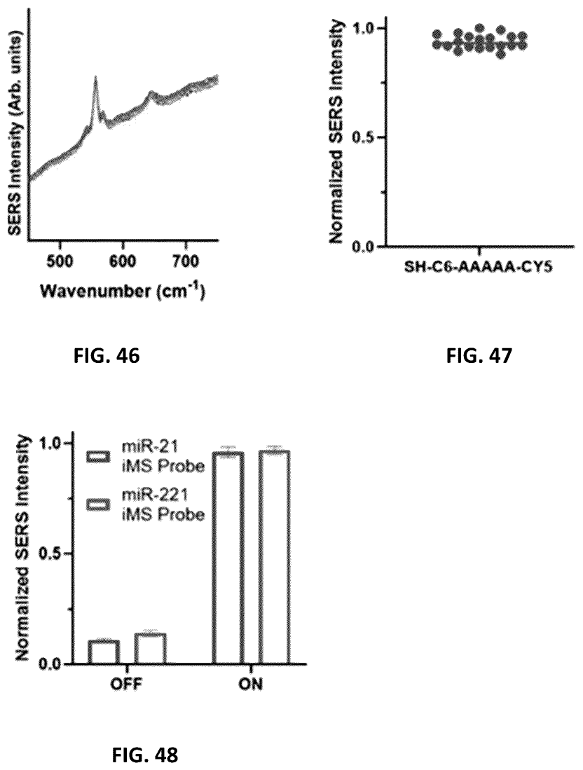

Finally, the diagnostic utility of the system was demonstrated through multiplexed detection of circulating CRC biomarkers from patient plasma samples. FIG. 26A depicts the sample workflow: enriched exosomal RNA from the acoustofluidic disc of the system is quantified and added to the nanostar-functionalized plasmonic biosensing unit integrated with iMS probes. The miRNA biomarkers miR-21 and miR-221 were chosen as targets because there is evidence that both sequences are significantly upregulated in patient plasma. FIG. 26B illustrates the process by which the multiplexed SERS spectra resulting from the system analysis undergo spectral unmixing for individual probe signal quantification. The oligonucleotide SH-C6-AAAAA-CY5 was used to test the repeatability and reproducibility of the SERS signal generated from the nanostar substrate. The spectra of 20 distinct sensing regions were recorded and analyzed, with representative raw spectra shown in FIG. 46. This resulted in an average peak height at 557 cm-1 after signal normalization of 0.940±0.033 (3.5%), shown in FIG. 47. Next, to demonstrate how the assay can be repeatable and reproducibly prepared, the SERS signal of 10 different sensing regions was recorded for each iMS probe at different stages throughout the assay preparation process. The normalized peak height at 557 cm-1 for the miR-21 probe and at 1461 cm-1 for the miR-221 probe in the “ON” and “OFF” configurations is shown in FIG. 48. After signal normalization, the average peak height for the miR-21 iMS probe in the “ON” configuration was 0.965±0.024 (2.5%) and 0.109±0.007 (6.4%) for the “OFF” configuration. The average peak height after normalization for the miR-221 iMS probe in the on configuration was 0.969±0.018 (1.8%) and 0.141±0.014 (9.9%) in the OFF configuration.

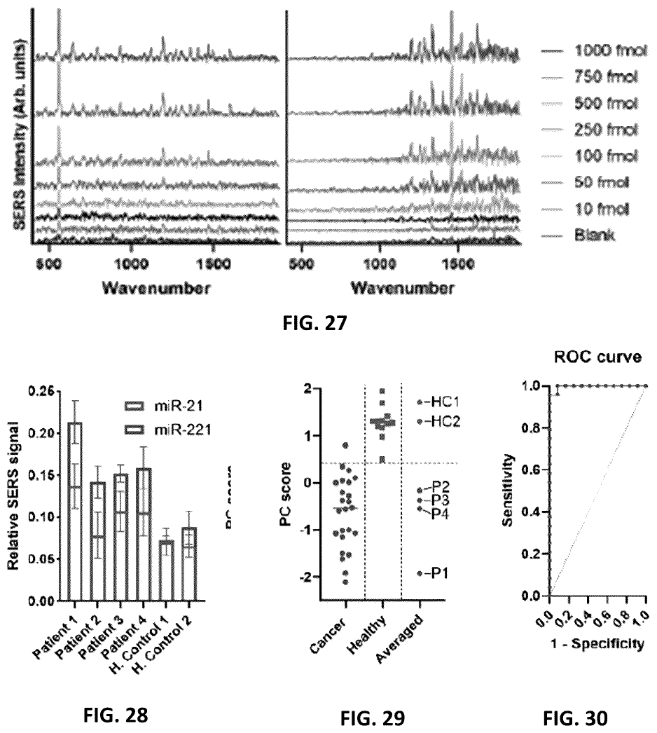

FIG. 27 shows representative changes in SERS intensity as a function of synthetic analyte added to the sensing region of the substrate for miR-21 and miR-221 iMS probes. Both iMS probes display highly linear behavior across the range of targets investigated, with R2 values of 0.9905 and 0.9748 for miR-21 and miR-221 when measured in triplicate. The limit of detection for miR-21 is 19 picograms and for miR-221 is 17 picograms. 20 ng aliquots of enriched small RNA obtained from the acoustically enriched patient samples were added to individual sensing regions of the plasmonic nanostar-based biosensing unit, which was integrated with both miR-21 and miR-221 iMS probes. Each patient sample was analyzed 6 times, and the signal contributions for each iMS probe are shown in FIG. 28. The samples positive for CRC had greater iMS probe signal contributions for both miRNA biomarkers compared to the control group.

To confirm the specificity of the analysis, the Pearson correlation coefficient between the mean 2^{-(ΔΔct)} values of each miRNA of interest and the corresponding iMS probe signal increase for each patient was analyzed, resulting in a Pearson correlation coefficient of 0.75 and 0.67 for miR-21 and miR-221. To assess the diagnostic performance of the ASCENDx analysis, the relative iMS probe signal increases for each patient were analyzed via principal component analysis (PCA), with results shown in FIG. 29. By establishing a threshold PC value at 0.4, 35 of 36 samples were completely partitioned by health status, resulting in a test with 95.8% sensitivity and 100% specificity. The iMS probe signal contributions for all samples were averaged per patient and were completely partitioned based on health status upon reanalysis. ROC curve analysis of all patient data is shown in FIG. 30, resulting in an area under the curve of 0.997 [0.985, 1.00] with 95.8% sensitivity and 91.7% specificity at a classification cutoff at 0.5. Adjusting the classification cutoff to 0.8 results in a diagnostic assay performance of 95.8% sensitivity and 100% specificity.

### Discussion

Circulating exosomal cargo in the form of oligonucleotides and proteins can provide insight into patient health status, including disease development and progression. However, the rate of biological research and clinical translation of exosomal cargo profiling is hindered by the difficulty of removing non-exosomal contaminants and concentrating the biomarkers of interest. Moreover, exosomes are often isolated and analyzed separately, further adding to the complexity and processing time required. The system provided herein addresses the shortcomings associated with traditional exosomal processing and subsequent miRNA detection.

The end-to-end patient sample analysis system provides exosome enrichment, on-disc label-free SERS verification of exosome enrichment, and the amplification-free multiplexed detection of miRNA biomarkers contained within that enriched sample. The miniature acoustofluidic disc is capable of isolating exosomes with high yield and purity from low-volume biofluid samples by applying SAWs coupled into a fluid droplet on which a disc is placed (Table 1). Plasmonic nanostar particles were optimized and integrated onto the surface of the acoustofluidic disc to facilitate label-free SERS verification of exosome enrichment after acoustic separation. RT-PCR results confirmed that the miRNA content of enriched exosome samples was more strongly correlated with health status compared to untreated plasma, providing a compelling example of how the system can readily be adapted for biomarker discovery applications. The plasmonic biosensing unit of the system effectively discriminated between acoustically enriched miRNA derived from CRC-positive patients and healthy controls with 95.8% sensitivity and 100% specificity, a rapid sample-to-answer time of 30 minutes, and was strongly correlated with RT-PCR results. Moreover, this is the first report of multiplexed, amplification-free direct detection of miRNA biomarkers from acoustically enriched patient plasma samples. The system workflow established herein acts as a proof-of-concept validation and demonstrates the synergy between these sample processing and biosensing platforms.

TABLE 1
Competitive analysis for different exosome isolation methods

| | Differential | Polymer | Membrane | Immunoaffinity | Disclosed |
|---|---|---|---|---|---|
| Parameter | Ultracentrifugation | precipitation | Affinity | Capture Beads | System |
| Yield (%) | 5-25% | ~40% | ~9% | ~30% | ~86% |
| Purity | 3 × 10⁵- | 6.1 × 10⁷- | ~1 × 10⁷ | ~2 × 10⁶ | ~3 × 10⁸ |
| (particles/pig | 4 × 10⁷ | 2 × 10⁸ | | | |
| of protein) | | | | | |
| Processing | 8-12 | ~30 min - | ~30 | ~6-24 | ~30 |
| Time | hours | overnight | minutes | hours | minutes |
| | | incubation | | | |
| Minimal | ~5 mL | ~1 mL | ~200 μL | ~500 μL | ~3 μL |
| Processing | | | | | |
| Volume | | | | | |

The system and method can be applied to various health status assessment applications because of the inherently versatile underlying technologies. Regardless of the exosomal biomarker of interest, all exosome-containing biofluids are eligible for enrichment. Easily accessible samples such as saliva are strong candidates for biomarker separation. While CRC was used to demonstrate the diagnostic advantage provided by the system and method, ailments ranging from several types of cancer, neurodegenerative diseases, and even acute injuries that cause circulating miRNA dysregulation are candidates for investigation. Finally, the flexibility provided by the system allows for the rapid design, validation, and integration of probes for any miRNA biomarker target.

In summary, the system described herein provides for rapid and effective sample enrichment and subsequent miRNA biomarker detection. The system significantly simplifies analytical protocols and unlocks exosome's transformative potential in biomedical research and diagnostic applications.

### Materials and Methods

The SPFTs were fabricated by depositing a 5-nm-thick layer of Cr and a 150-nm-thick layer of Au onto a 128° Y-cut LiNbO3 wafer using electron beam evaporation. The photoresist patterns on the LiNbO3 wafer used for the metal evaporation were fabricated via photolithography, and the excess metal was removed using a standard lift-off process with acetone. All SPFTs were composed of 116 pairs of electrode fingers. Each SPFT resulted in a SAW frequency of 30 MHz. Using silver epoxy, external wires were bonded to the electrodes. A biopsy punch created the PDMS ring that confines the droplet. The PDMS ring and LiNbO3 substrate were treated with an oxygen plasma to promote surface bonding, followed by a post-bake at 65° C. for 8 hours. A function generator and an amplifier activated the SPFTs and generated SAWs. The PDMS discs were fabricated using standard SU-8 soft lithography and the PDMS mold-replica process. Discs can be left open or covered by a thin membrane to prevent sample evaporation or ambient contamination. All experiments described herein were performed using an enclosed disc.

### Image Acquisition and Analysis

The images were acquired using an upright microscope and a charge-coupled device camera. The disc's rotational speed was measured using a smartphone camera with 30-240 fps. The data and figures were analyzed using ImageJ. The nanoparticle size distribution was analyzed using an NS500 running NTA software 3.4 by Malvern Panalytical.

### Protein Quantification

Proteins were quantified using a bicinchoninic acid (BCA) protein concentration assay kit. To establish a quantification curve, 10 μL of BSA solution (0, 0.025, 0.075, 0.125, 0.250, 0.500, 0.750 and 1.000 mg/mL) was added to the wells of a microtiter plate. Then, 200 μL BCA working solution was added to the wells and incubated at 37° C. for 30 min. Absorbance was measured using a BioTek Synergy H1 Multimode Reader.

### Synthesis of Bimetallic Nanostars

To prepare 100 mL of bimetallic nanostar solution, 1 mL of 10 nM 12 nm gold seed solution was added to 100 mL of 25 mM HAuCl4 and 100 μL of HCl. Next, 500 μL of 6 mM AgNO3 was added to the mixture, followed rapidly by 500 μL 0.1M ascorbic acid and then between 150 and 1000 μL 0.1M AgNO3 depending on the type of bimetallic nanostar. 100 μL of 30% ammonia solution was then added to terminate the reaction.

### Substrate Fabrication

Bimetallic nanostar substrates were prepared using 8 mm in diameter coverslips in the case of “on-disc” SERS measurements, and 25 mm×75 mm microscope slides in the case of the miRNA assay. All substrates were cleaned using piranha solution for 1 hour, washed in DI water, and dried overnight at 110° Celsius. Substrates were then placed into sealed vapor deposition chambers with 50 μL N-(2aminoethyl)-3-aminoproyltriethoxysaline and left in an oven for 48 hours at 90° Celsius. Once vapor deposition was complete, substrates were washed in toluene, ethanol, and DI water to remove non-covalently linked aminosilane on the substrate surface. Substrates were then submerged into a bimetallic nanostar solution for 3 days, at which point they were removed, rinsed with DI water, and stored under nitrogen until further use.

### SOMSOL Simulation of Bimetallic Nanostars

The wave optics package in COMSOL Multiphysics 6.0 simulates the magnitude of local electric field enhancement achieved by different morphologies of bimetallic nanostar particles. Optical parameters described by Rakic et al. were used for gold and silver in this model. The surroundings were modeled as water, using the optical parameters described by Daimon and Masamura. The entire model was meshed using the extremely fine setting defined by the pre-determined conditions in the wave optics module.

### SERS Measurements on the Disc of the System

Measurements were recorded using a Horiba Jobin Yvon LabRam ARAMIS system. The excitation wavelength used for this study was 633 nm at 50% power. Spectra were recorded for 10 seconds and 3 accumulations each. All spectra were recorded using a 100× objective.

### Collection of Patient Samples

Plasma samples were stored at −80° C. until testing. Samples were processed as part of standard care for patients with excess plasma not needed for testing banked frozen. A Peltier cooling plate was placed under the device during blood and plasma separation experiments to reduce heating and maintain sample integrity.

miRNA Isolation from Patient Samples

miRNA was isolated from patient plasma or spun samples using the commercially available mirVANA PARIS kit, using the manufacturer's protocol. For each sample, 250 μL was added to an equal volume of 2× denaturing solution, followed by 500 μL acid phenol. After homogenization, samples were centrifuged at 11,000 g for 5 minutes. The entire aqueous phase was then collected and mixed with ⅓ volume of ethanol and added to a tube containing a glass fiber filter. The sample was then passed through the filter, and the flowthrough was collected. To enrich the sample for small RNAs, ⅔ volume of ethanol was added to the flow through and passed through a fresh glass fiber filter. This filter was then washed with the included miRNA washing buffers as indicated by the manufacturer's protocol. The enriched small RNA was eluted with 50 μL DI water. Nucleotide quantification was done using a Nanodrop OneC.

cDNA Synthesis

The isolated small RNA from each patient was used to synthesize cDNA for future RT-PCR analysis. cDNA was prepared using the miRCURY LNA RT kit, following all manufacturer protocols. A reverse transcriptase master mix was prepared with 5× miRCURY RT SYBR Green Reaction Buffer, 10× miRCURY RT Enzyme Mix, UniSP6 RNA spike-in control, and RNase-free water. 10 ng of isolated small RNA was added to each reverse transcription reaction. All reverse transcription reactions were incubated for 60 minutes at 42° C. and then at 95° C. for 5 minutes. RT reaction mixtures were then immediately cooled to 4° C. until further use.

### Substrate Assembly

To fabricate the well system onto the surface of the nanostar substrate, a Cricut cutting machine was used to cut double-sided adhesive from 3M into squares with an edge length of 7 mm, and with a hole 4 mm in diameter removed from the center. This adhesive was used to mount PDMS wells with the same dimensions and 4 mm in height onto the surface of the substrate.

### iMS Probe Oligonucleotide Sequences

The sequence of the miR-21 probes, placeholder, and synthetic target used in this study were 5′-SH-AAAAAGTCTGTATTAAAAAATAGCTTATCAGAC-Cy5-3′, 5′-TCAACATCAGTCTGATAAGCTATTTT-3′, and 5′-TAGCTTATCAGACTGATGTTGA-3′, respectively. The sequence of the miR-221 iMS probes, placeholder, and synthetic target used in this study were 5′-SH-AAAAAGCAGAATTAAAA AAAAAAGCTACATTGTCTGC-cy3-3′, 5′-GAAACCCAGCAGACAATGTAGCTTTTT-3′, and 5′-AGCTACATTGT CTGCTGGGTTTC-3′, respectively.

### RT-PCR

RT-PCR was performed using the miRCURY LNA SYBR® Green PCR Kit and miRCURY® LNA® miRNA PCR Assays. The PCR assays used in this study were designed to detect hsa-miR-21-5p, hsa-miR-221-3p, and the UniSp6 spike-in control. cDNA templates from the RT reactions were all diluted at 1:30 prior to their addition. A master mix containing 2× miRCURY SYBR Green Master Mix, 1 μL PCR primer mix, and 1 μL RNase-free water was prepared for each of the three targets of interest. Each target was run in triplicate for each patient sample using a Roche LightCycler 480 real-time cycler. Run conditions were set according to the miRCURY® LNA® miRNA PCR Assays Quick-Start Protocol. Relative expression was evaluated via the 2−(ΔΔCp) method. All samples were analyzed in triplicate.

### iMS Assay Preparation

The iMS assay for both miR21 and miR221 iMS probes, 1 μM probe in 1×PBS and 0.01% Tween 20 and 100 μM tris(2-carboxyethyl)phosphine (TCEP) was incubated for 3 hours. In each substrate well, 3 μL of iMS probe TCEP solution, 4.5 μL of 100 μM mercaptohexanol (MCH) solution diluted in 1×PBS and 0.02% Tween 20, 12 μL of 1×PBS and 0.02% Tween 20 and 15 μL 20×PBS. After loading, the substrate was placed on a shaker overnight. All wells were then washed using 1×PBS and 0.01% Tween 20 solution. Next, 30 μL of 100 μM MCH was added to each well, and the substrate was placed in a heated shaker set to 35° C. for 30 minutes. All wells were then washed with 1×PBS and 0.01% Tween 20 solution. After washing, 30 μL of 3 μM placeholder solution was added to each well and placed into a heated shaker for 1 hour at 45° C. Substrate wells were then washed for a final time, and blank signals were then recorded for all wells to ensure the iMS assay was correctly prepared in all wells. For each patient, 20 ng of total small RNA was added to a PBS and Tween 20 mixture, resulting in patient sample solution containing a total of 20 ng small RNA in 1×PBS and 0.01% Tween 20 with a total volume of 10 μl. Patient sample solutions were added to the prepared substrate wells and allowed to react for 30 minutes. All spectra were recorded from regions of the substrate with no prior laser exposure.

### SERS Signal Collection and Analysis

All iMS assay measurements were performed using a Renishaw InVia confocal Raman microscope equipped with a 632.8-nm HeNe laser and a 10× objective. Before each measurement, the laser spot was focused onto the surface of the substrate to ensure that the same amount of substrate area was exposed to the laser during every measurement. Using ImageJ, the area of this laser spot was measured and used to calculate the absolute mass of the target within the sensing region.

All recorded spectra were processed in MATLAB, where they were background subtracted and smoothed with a Savitzky-Golay filter (five-point window and first-order polynomial). For the analysis of multiplexed spectra, the spectral decomposition developed by Lutz et al. was used to determine to what amount each individual iMS probe signal contributed to the recorded spectra. This method uses reference spectra for the individual miRNA probes, the multiplexed blank signal of each well prior to addition of the patient sample solution, and the multiplexed signal after the addition of patient sample. After removing the signal contribution from the multiplexed blank spectra, the MATLAB functions Isqnonnneg and fmincon, which solve non-negative least-squares curve fitting problems and minimize a system of nonlinear equations respectively, are used to determine the contribution each reference spectra to the patient sample signal. This analysis generates minimally constrained coefficients by which the reference spectra are multiplied to produce the best fit to the input patient spectra. These coefficients then allow for the direct comparison between patient spectra for each individual iMS probe signal.

### Example 2

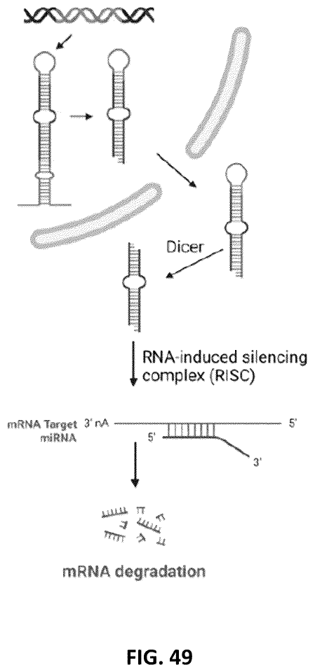

FIG. 49 illustrates the function of microRNA (miRNA). When fully matured, miRNA are single-strand RNA sequences, most often between 21 and 25 nucleotides in length. miRNA negatively regulates the translation of proteins through triggered degradation of mRNA, induced by partially complementary bonding.

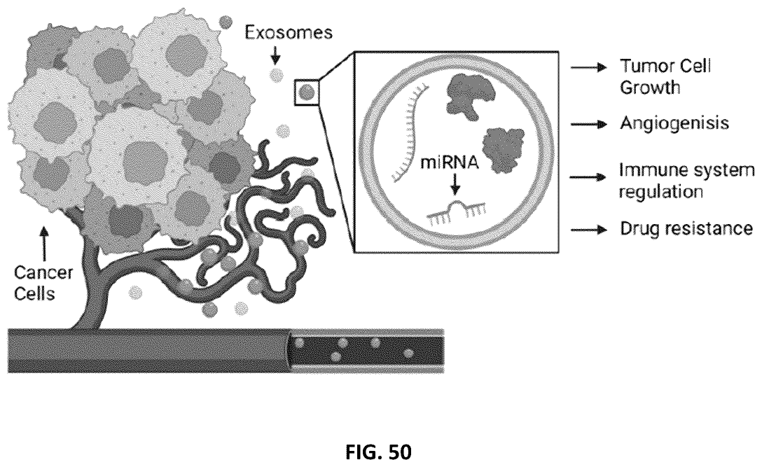

FIG. 50 illustrates miRNA as an attractive option as a disease biomarker. Some cancer cells release exosomes with oncogenic cargo that promotes local tumor cell growth, angiogenesis, drug resistance, and alterations in the immune system. These exosomes exit the tumor micro-environment and enter the bloodstream.

Disease detection via miRNA profile in biofluid has been attempted using traditional methods. The exosomes derived from cancer cells that enter circulation often have far different miRNA profiles compared to normal circulating exosomes. This difference has been detected in biofluids such as plasma, serum, and saliva from patients with several types of cancer. For example, the following cancers have been detected using miRNA profiling: breast, lung, ovarian, colorectal, gastric, prostate, bladder, liver, and kidney. The system described herein can more accurately and efficiently detect all of these cancers. Further, due to the mobility and ease at which the system described herein can be used, diagnosis of diseases and disorders can decrease turnaround times and costs for diagnosis.

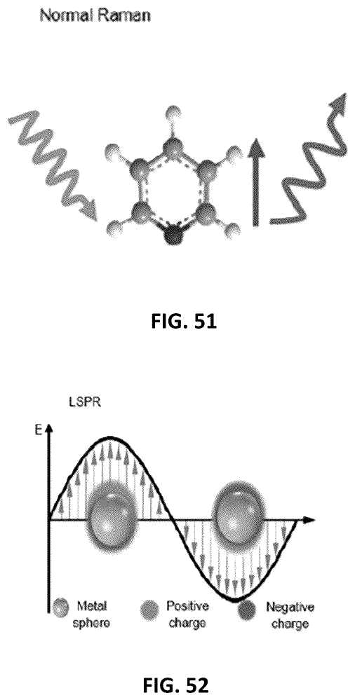

FIG. 51 illustrates a normal Raman scattering approach where inelastic scattering of light corresponding to the energy of vibrational modes result in a change of polarizability.

FIG. 52 illustrates metal spheres (e.g., the plasmonic nanostars described herein) which can be used to concentration light, creating intense hotspots that amplify weak Raman signals of nearby molecules, thereby enabling ultrasensitive detection and analysis, known as SERS.

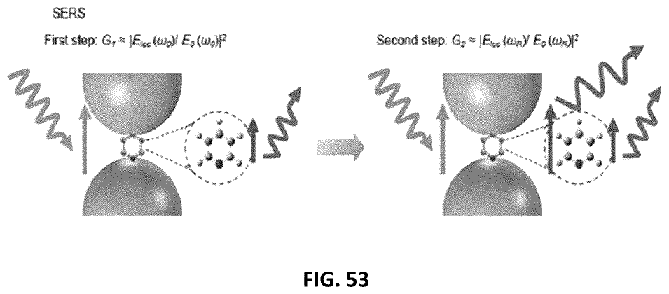

FIG. 53 illustrates SERS, which uses high local electric field enhancement generated by metallic nanostars to improve Raman intensity.

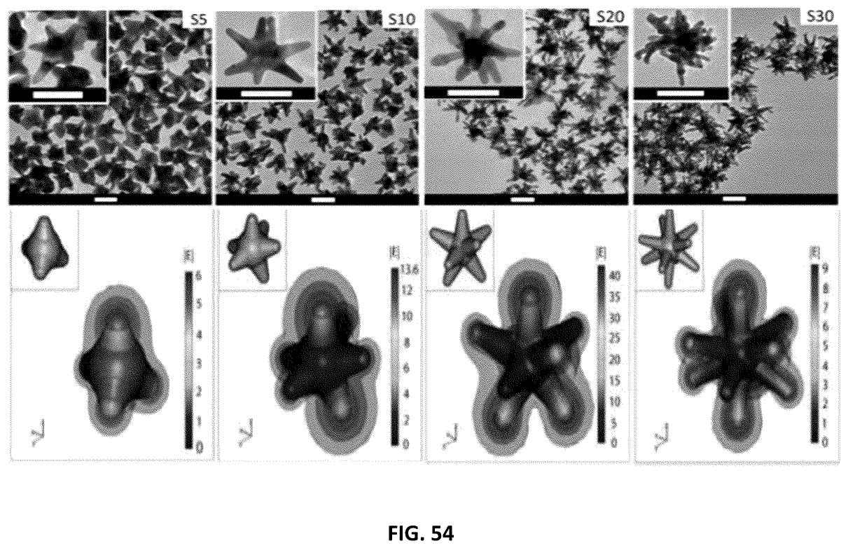

FIG. 54 illustrates four nanostars used as a nanoplasmonic platform to leverage high aspect ratio branches to generate an intense local electric field enhancement. Red color indicates a high local electric field enhancement while blue illustrates a lower electric field enhancement.

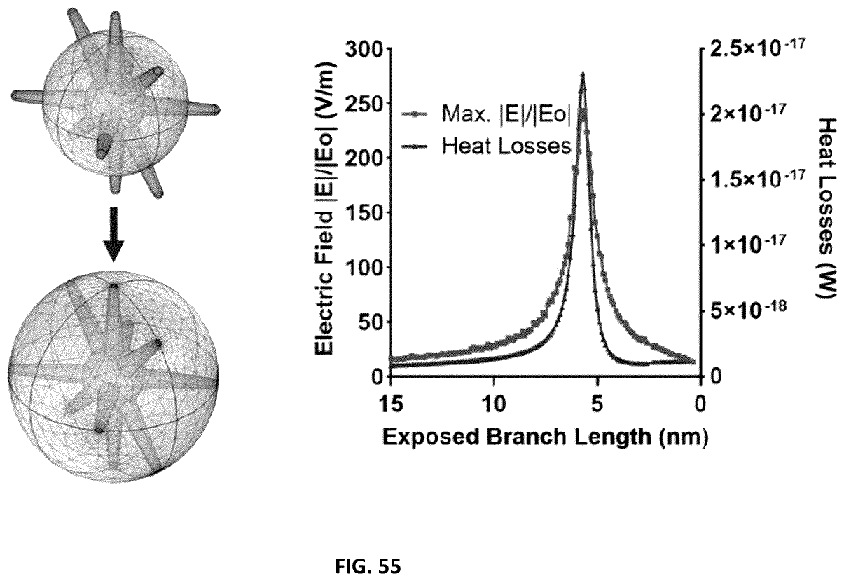

FIG. 55 illustrates the theoretical enhancement of bimetallic nanostars described herein. For example, as illustrated, the electric field increases most significantly when the exposed branch length of the nanostars (e.g., gold branches exposed outside of the silver shell) is about 5 nm.

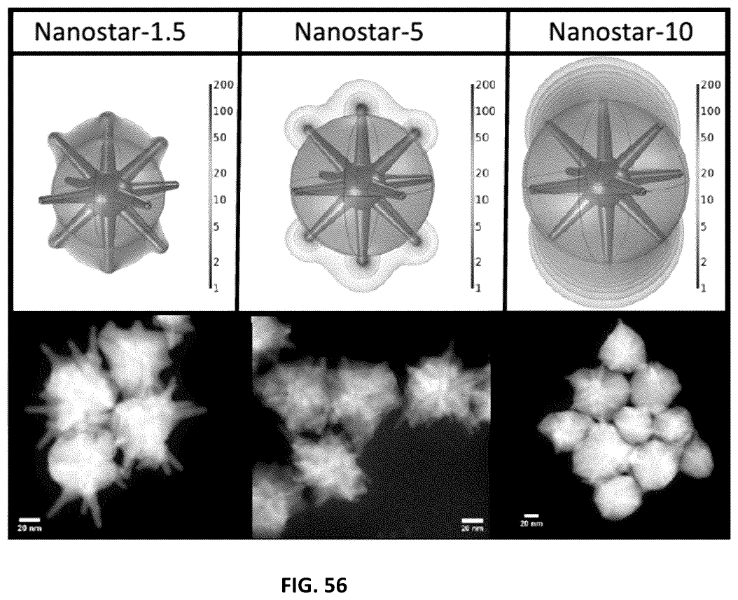

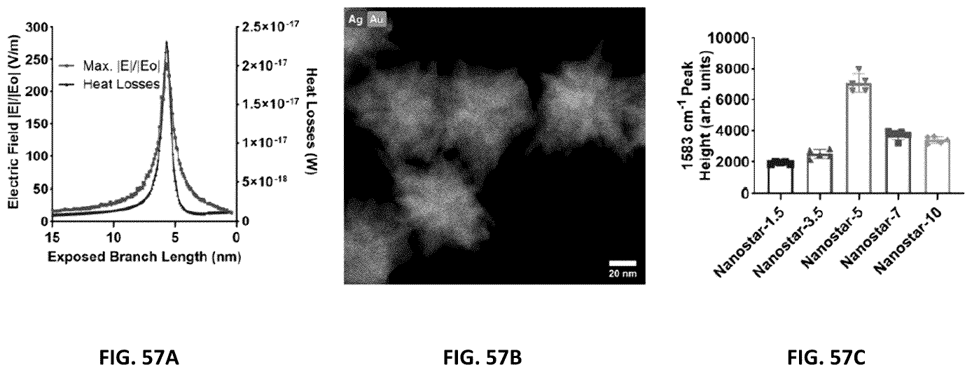

FIG. 56 illustrates the various nanostars described herein and the corresponding HAADF-STEM images. FIGS. 57A-57C illustrate the experimental validation of the various nanostars described herein.

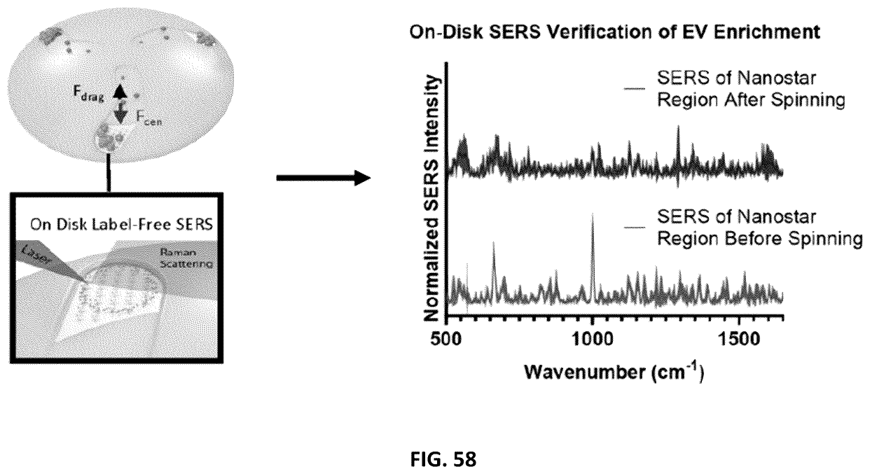

FIG. 58 illustrates another example of the label-free exosome enrichment verification described herein.

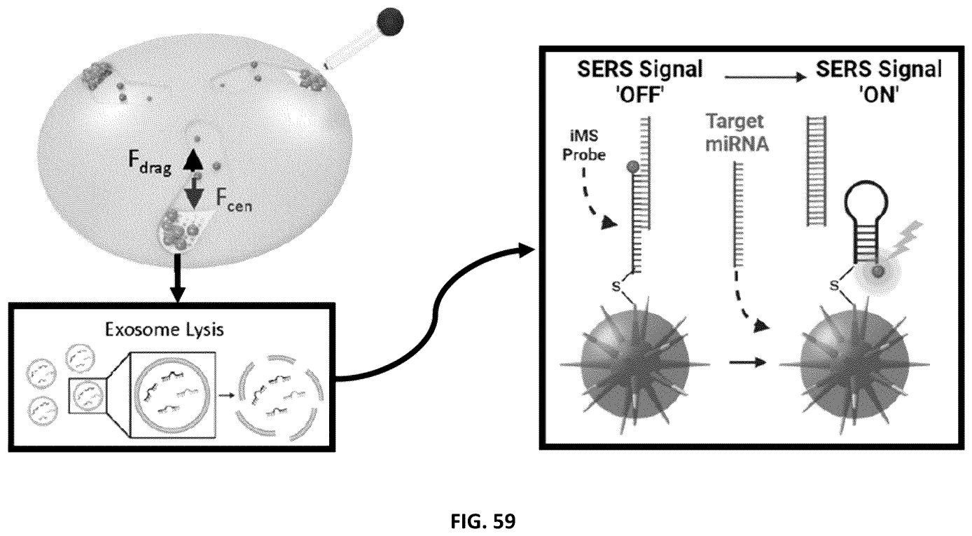

FIG. 59 illustrates another example of the SERS-based liquid biopsy of enriched exosomes described herein.

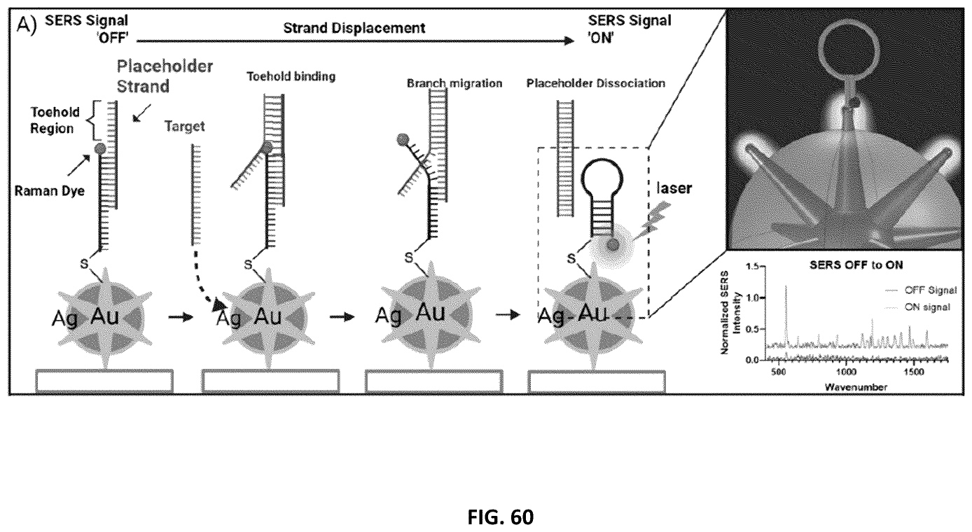

FIG. 60 is a schematic diagram of the inverse molecular sentinel miRNA probes described herein. For example, FIG. 60 illustrates the inverse molecular sentinel miRNA probe attaching to the target and the corresponding normalized SERS intensity signal.

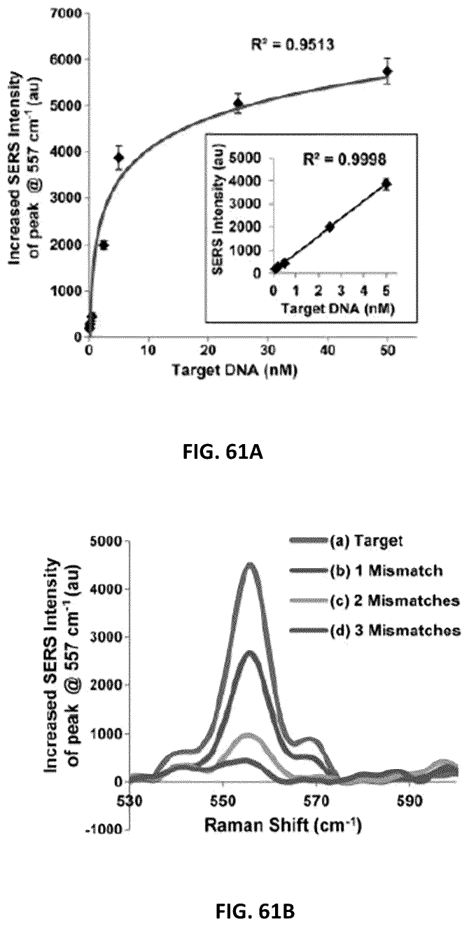

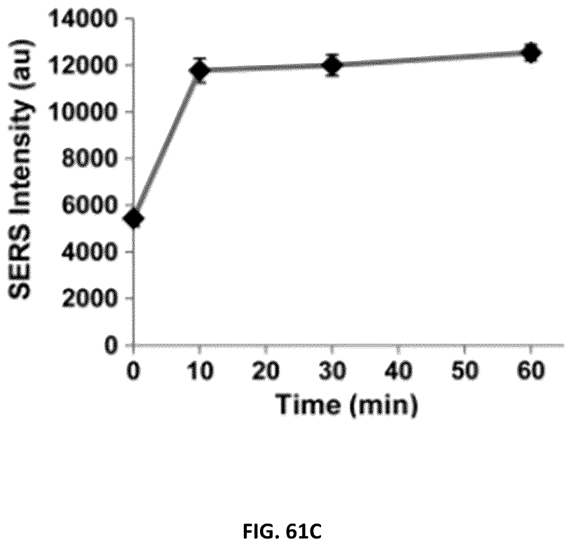

FIG. 61A illustrates the increased SERS intensity and the target DNA (nM). The linear dynamic range is between 1 and 10 nM. LOD can be calculated to be 0.1 nM/200 amol.

FIG. 61B illustrates that the SERS signal dramatically decreases as the number of mismatches between the placeholder and the target sequence increase.

FIG. 61C illustrates that the target miRNA sensing can be completed in less than 10 minutes.

The disclosures shown and described above are only examples. Even though numerous characteristics and advantages of the present technology have been set forth in the foregoing description, together with details of the structure and function of the present disclosure, the disclosure is illustrative only, and changes may be made in the detail, especially in matters of shape, size and arrangement of the parts within the principles of the present disclosure to the full extent indicated by the broad general meaning of the terms used in the attached claims. It will therefore be appreciated that the examples described above may be modified within the scope of the appended claims.

What is claimed is:

1. A system for separation of particles in a liquid sample, the system comprising:
a substrate;
a containment structure disposed on the substrate and defining a fluid-retaining region configured to contain a fluid;
one or more acoustic transducers coupled to the substrate and configured to provide acoustic waves to the fluid such that the fluid is rotated by the acoustic waves; and
a disc comprising a plurality of microfluidic channels, the disc configured to be disposed on top of the fluid and rotate with the fluid, the plurality of microfluidic channels configured to receive the liquid sample,
wherein rotation of the disc establishes a centrifugal field and an induced fluid flow within the plurality of microfluidic channels to transport particles within the liquid sample along the plurality of microfluidic channels at velocities dependent on one or more of particle size and particle density, thereby effecting separation and/or enrichment of a selected particle fraction of the liquid sample.

2. The system of claim 1, wherein the plurality of microfluidic channels are slanted channels having a first portion extending to a second portion, wherein the first portion is connected to the second portion at an angle, wherein the angle is about 15 degrees to about 90 degrees, and wherein the angle shortens a particle transport path and increases a separation rate of the particles in the liquid sample.

3. The system of claim 1, further comprising at least one biosensing unit located along at least one of the plurality of microfluidic channels, wherein the at least one biosensing unit is configured to determine a composition of the liquid sample at a location along at least one of the plurality of microfluidic channels.

4. The system of claim 3, wherein the at least one biosensing unit comprises:
a surface-enhanced Raman spectroscopy region including a plurality of plasmonic nanostructures immobilized at the location along at least one of the plurality of microfluidic channels, the plurality of plasmonic nanostructures configured to provide electromagnetic field enhancement sufficient to detect target-induced spectral signatures; and
an excitation light source configured to illuminate the surface-enhanced Raman spectroscopy region at an excitation wavelength sufficient to induce a reporter Raman signal,
wherein the reporter Raman signal is indicative of the composition of the liquid sample in the surface-enhanced Raman spectroscopy region.

5. The system of claim 4, wherein the at least one biosensing unit further comprises a plurality of nucleic acid or nucleic acid analog probes covalently attached to the plurality of plasmonic nanostructures and configured as inverse molecular sentinel probes, that, upon hybridization with one or more biomarkers in the liquid sample, undergo a conformational change that increases the reporter Raman signal.

6. The system of claim 5, wherein the plurality of nucleic acid or nucleic acid analog probes comprise multiplexed inverse molecular sentinel probes specific for at least one microRNA target.

7. The system of claim 4, wherein the plurality of plasmonic nanostructures comprise bimetallic nanostars each having a gold core and a silver shell with a shell thickness configured to maximize the electromagnetic field enhancement.

8. The system of claim 1, wherein the one or more acoustic transducers comprise two opposed pairs of acoustic transducers, each pair positioned on opposing sides of the fluid-retaining region.

9. The system of claim 1, wherein the selected particle fraction includes exosomes.

10. The system of claim 1, wherein the containment structure comprises a hydrophobic ring bonded to the substrate.

11. The system of claim 1, wherein the disc further comprises a membrane configured to enclose the liquid sample within the plurality of microfluidic channels, wherein the membrane is configured to reduce evaporation and ambient contamination.

12. The system of claim 1, further comprising a temperature control element thermally coupled to the substrate and configured to maintain a sample temperature within a predetermined range.

13. A method for separation of particles in a liquid sample, the method comprising:
providing the liquid sample to a plurality of microfluidic channels of a disc;
setting the disc on top of a fluid contained within a fluid-retaining region of a containment structure disposed on a substrate; and
providing, via one or more acoustic transducers coupled to the substrate, acoustic waves to the fluid such that the fluid is rotated by the acoustic waves, thereby rotating the disc,
wherein rotation of the disc establishes a centrifugal field and an induced fluid flow within the plurality of microfluidic channels to transport particles within the liquid sample along the plurality of microfluidic channels at velocities dependent on one or more of particle size and particle density, thereby effecting separation or enrichment of a selected particle fraction of the liquid sample.

14. The method of claim 13, further comprising providing, via an excitation light source, an excitation light to a surface-enhanced Raman spectroscopy region along at least one of the plurality of microfluidic channels to induce a reporter Raman signal, wherein the reporter Raman signal is indicative of a composition of the liquid sample in the surface-enhanced Raman spectroscopy region.

15. The method of claim 14, wherein the surface-enhanced Raman spectroscopy region comprises a plurality of plasmonic nanostructures configured to provide electromagnetic field enhancement sufficient to detect target-induced spectral signatures.

16. The method of claim 15, further comprising detecting an increase to the reporter Raman signal when a plurality of nucleic acid or nucleic acid analog probes covalently attached to the plurality of plasmonic nanostructures hybridize with one or more biomarkers in the liquid sample and undergo a conformational change.

17. The method of claim 16, wherein the plurality of nucleic acid or nucleic acid analog probes comprise multiplexed inverse molecular sentinel probes specific for at least one microRNA target.

18. The method of claim 17, wherein the plurality of nucleic acid or nucleic acid analog probes comprise oligonucleotide probes and the at least one microRNA target comprises miR-21 and miR-221.

19. The method of claim 18, wherein the at least one microRNA target is indicative of circulating colorectal cancer.

20. The method of claim 13, further comprising providing, via a temperature control element thermally coupled to the substrate, temperature regulation to maintain a sample temperature within a predetermined range.
